(19) United States
(12) Patent Application Publication
(10) Pub. No.: US 2009/0156465 A1
(43) Pub. Date: Jun. 18, 2009

(54) DERIVATIVES OF BETA-AMINO ACID AS DIPEPTIDYL PEPTIDASE-IV INHIBITORS

(76) Inventors: Jitendra A. Sattigeri, Gurgaon (IN); Shahadat Ahmed, Gurgaon (IN); Murugaiah M.S. Andappan, Pudukottai (IN); Sachin Sethi, Yamuna Nagar (IN); Lalima Sharma, Chandigarh (IN); Chanchal Kumar Pal, Midnapur (IN); Sachin Ramesh Kandalkar, Ahmednaga (IN); Dipak C. Mahajan, Jalgaon (IN); Kaushal Kishore, Nalanda (IN); Sumati Bhatia, Delhi (IN); Anil G. Gadhave, Ahmednagar (IN); Vinay S. Bansal, New Delhi (IN); Joseph Alexanand Davis, Trichirappali (IN)

Correspondence Address: RANBAXY INC., 600 COLLEGE ROAD EAST, SUITE 2100, PRINCETON, NJ, 08540, US

(21) Appl. No.: 12/159,562

(22) PCT Filed: Dec. 12, 2006
(86) PCT No.: PCT/IB06/55006

§ 371 Date: Feb. 24, 2009

(30) Foreign Application Priority Data
Dec. 30, 2005 (IN) 3520/DE/2005

(51) Int. Cl. A61K 38/28; C07D 413/02; A61K 31/5377; C07D 241/36; A61K 31/4995; A61K 31/702; A61K 31/397; A61P 3/00; A61P 3/10; A61P 37/00; A61P 35/00; A61P 1/00; A61P 15/08

(52) U.S. Cl. 514/4; 544/129; 514/235.5; 544/349; 514/249; 514/25; 514/210.02

(57) ABSTRACT

The present invention relates to β-amino acid derivatives as dipeptidyl peptidase-IV inhibitors and the processes for the synthesis of the same. This invention also relates to pharmacological compositions containing the compounds of the present invention, and methods of treating diabetes, especially type 2 diabetes, as well as prediabetes, diabetic dyslipidemia, metabolic acidosis, ketosis, satiety disorders, and obesity. These inhibitors can also be used to treat conditions manifested by a variety of metabolic, neurological, anti-inflammatory, and autoimmune disorders like inflammatory disease, multiple sclerosis, rheumatoid arthritis; viral, cancer and gastrointestinal disorders. The compounds of this invention can also be used for treatment of infertility arising due to polycystic ovary syndrome.

### FIELD OF THE INVENTION

The present invention relates to β-amino acid derivatives as dipeptidyl peptidase-IV inhibitors and the processes for the synthesis of the same. This invention also relates to pharmacological compositions containing the compounds of the present invention, and methods of treating diabetes, especially type 2 diabetes, as well as prediabetes, diabetic dyslipidemia, metabolic acidosis, ketosis, satiety disorders, and obesity. These inhibitors can also be used to treat conditions manifested by a variety of metabolic, neurological, anti-inflammatory, and autoimmune disorders like inflammatory disease, multiple sclerosis, rheumatoid arthritis; viral, cancer and gastrointestinal disorders. The compounds of this invention can also be used for treatment of infertility arising due to polycystic ovary syndrome.

### BACKGROUND OF THE INVENTION

Type 2 diabetes mellitus, also known as “non-insulin dependent diabetes mellitus” (NIDDM), afflicts an estimated 6% of the adult population in western society and is expected to grow at a rate of 6% per annum worldwide. Type 2 diabetes is a complex metabolic disorder, characterized by hyperglycemia and hyperinsulinemia. This results from contribution of impaired insulin secretion from β-cells in pancreas and insulin resistance, mainly, in muscle and liver. The insulin resistant individuals, in addition to being hyperglycemic, exhibit a constellation of closely related clinical indications, which include obesity, hypertension and dyslipidemia. The uncontrolled hyperglycemia can further lead to late-stage microvascular and macrovascular complications such as nephropathy, neuropathy, retinopathy and premature atherosclerosis. In fact, 80% of diabetic mortality arises from atherosclerotic cardiovascular disease (ASCVD).

Presently, several pharmacological agents are available as antihyperglycaemic agents to mitigate the conditions manifested in NIDDM (Lancet, (2005) 365, 1333-1346). These include (1) insulin secretagogues, which increase insulin secretion from pancreatic cells [e.g., sulphonyl ureas (glimeperide) and non-sulphonyl ureas (repaglinide)], (2) biguanides, which lower hepatic glucose production, e.g., metforminutes, and (3) α-glucosidase inhibitors, which delay intestinal absorption of carbohydrates, e.g., acarbose (Lancet, (2005) 365, 1333-1346). The insulin sensitizers like pioglitazone and rosiglitazone (TZDs), which exhibit their effect by PPARγ agonism, control hyperglycaemia by improving peripheral insulin sensitivity without increasing circulating insulin levels. However, all these agents are associated with one or more of side effects like hypoglycaemia, gastrointestinal side effects including abdominal discomfort, bloating, flatulence, hepatotoxicity, weight gain, dilutional anemia and peripheral edema (Endocrine Rev., (2000) 21, 585-618).

Given its prevalence and complexity of NIDDM, there is a growing need for novel strategies and effective therapeutic approaches for treatment of diabetes. The safe and, preferably, orally bioavailable therapeutic agents, that would accelerate glucose clearance by stimulating endogenous insulin secretion in a glucose-dependent manner without hypoglycemic episodes and previously mentioned side effects, would represent an important advance in the treatment of this disease.

One such novel approach appearing on the horizon involves enhancing the levels of incretin (insulin-secreting) hormones, glucagon-like peptide-1 (GLP-1) and glucose-dependent insulinotropic polypeptide (GIP) (Expert Opin. Investig. Drug, (2005) 14, 57-64). These hormones mediate the process of insulin release from pancreatic β-cells in a glucose-dependent manner. GLP-1 (7-36) is a polypeptide of 29 amino acids derived by post translational processing of proglucagon in the L-cells of the distal small intestine in response to the food intake. It has multiple synergistic antidiabetic actions including stimulation of insulin secretion, inhibition of glucagon, inducement of feeling fullness and delayed gastric emptying. Administration (continuous infusion) of exogenous GLP-1 in diabetic patients has been demonstrated to be efficacious in lowering blood glucose levels by enhancing glucose-mediated insulin secretion, suppressing glucagon secretion and slowing gastric emptying. Additionally, preclinical studies with GLP-1 or Exendin-4 in streptozotocin-injected neonatal rats have implicated the role of GLP-1 in neogenesis and preservation of β-cells (Current Opin. Pharma., (2004) 4, 589-596; Expert Opin. Investig. Drug, 2003, 12, 87-100).

However, these incretin hormones are very short lived (t1/2 GLP-1=˜2 minutes, t1/2 GIP=˜7 min) because they are very rapidly cleaved by the enzyme dipeptidyl peptidase-IV (DPP IV, CD26, EC 3.4.14.5) to GLP-1 (9-36) and GIP (3-42), which are the weak antagonists of GLP-1 and GIP receptors respectively (Reg. Peptides, (2005) 128, 125-134). DPP IV is a serine protease known for cleavage of polypeptides with specificity for Pro/Ala at the penultimate position from the N-terminus. It is expressed on the surface of epithelial cells of intestine, liver, kidney proximal tubules, prostrate, corpus luteum, lymphocytes and macrophages. It is now proven that DPP IV inhibition leads to an increase of biologically active forms of both GLP-1 and GIP to therapeutically beneficial levels and thus enhances the body's own normal homeostatic mechanism. As the incretins are released by the body, only in response to the food intake, DPP IV inhibition is not expected to increase the level of insulin at inappropriate times, such as in between meals, which can otherwise lead to hypoglycemia. The initial proof of concept for DPP IV based therapy has been obtained from DPP IV knockout (KO) mice and other preclinical animal models. The DPP IV KO rat and mice have shown normal glucose tolerance and didn't develop diabetic symptoms, even when fed with fat-rich food. Clinical and pre-clinical studies with DPP IV-resistant GLP-1 analogs like Exenatide have provided indirect but valuable additional validation for the DPP IV target. In clinical trials with an early DPP IV inhibitor, viz., NVP DPP 728, significant improvement in mean 24 h glucose excursion with lower insulin, glucagon and HbAlc levels were observed in the treated patients. Experimental evidence suggests that DPP IV inhibition offers an added benefit in preservation and regeneration of 0 cells. DPP IV inhibitors may thus be used in disease modifying therapy in type 1 and late-stage type 2 diabetes.

GLP-1 has been proposed to be one of the physiological regulators of appetite and food intake. The DPP IV inhibitors may also manifest the beneficial effect of delaying gastric emptying observed with GLP-1. This is further corroborated by recent Phase II studies that no body weight gain was observed with DPP IV inhibitors during the treatment period of the patients with diabetes and obesity (Current Opin. Pharma., (2004) 4, 589-596).

The present invention provides DPP IV inhibitors and methods for treating conditions mediated by DPP IV like diabetes, especially, type 2 diabetes mellitus, as well as prediabetes, diabetic dyslipidemia, metabolic acidosis, ketosis, satiety disorders, and obesity. These inhibitors can also be used to treat conditions manifested by a variety of metabolic (Expert Opin. Investig. Drug, (2003) 12, 87-100), neurological (Brain Res., (2005) 1048, 177-184), anti-inflammatory, and autoimmune disorders (Clin. Diagnostic Lab. Immunol. (2002) 9, 1253-1259) like inflammatory disease, multiple sclerosis, rheumatoid arthritis (Clin. Immunol. Immunopath., (1996) 80, 31-37); viral (Clin. Immunol., (1999) 91, 283-295), cancer (Cancer Res., (2005) 65, 1325-1334), blood disorders (Blood, (2003) 102, 1641-1648) and gastrointestinal disorders. The compounds of this invention can also be used for treatment of infertility arising due to polycystic ovary syndrome.

WO 04/009544 discloses 2-cyano-4-fluoropyrrolidine derivatives or their salts. WO 03/106456 discloses compounds allegedly possessing dipeptidyl peptidase-IV enzyme inhibitory activity. WO 03/074500 discloses compounds which contain fluorine atoms and are said to be DPP IV enzyme inhibitors. WO 03/02553 discloses fluoropyrrolidines described as dipeptidyl peptidase inhibitors. WO 03/037327 discloses N-(substituted)pyrrolidine derivatives described as dipeptidyl peptidase-IV inhibitors. WO 03/057666 discloses inhibitors of dipeptidyl peptidase-IV. WO 01/055105 discloses N-(substituted)-2-cyanopyroles and pyrrolines, which are the inhibitors of the enzyme DPP IV. U.S. Pat. No. 6,011,155 discloses N-(substituted glycyl)-2-cyanopyrrolidines, pharmaceutical compositions containing them and their use in inhibiting dipeptidyl peptidase-IV. The compound (2S)-1-[[(3-hydroxy-1-adamantyl)amino]acetyl]-2-cyanopyrrolidine [vildagliptin] has been disclosed as a potent, selective, and orally bioavailable dipeptidyl peptidase-IV inhibitor with antihyperglycemic properties vide reference J. Med. Chem., (2003) 46(13), 2774-2789

WO 03/000181, WO 03/004498, WO 03/082817, WO 04/007468, WO 04/0167133, WO 04/032836, WO 04/037169, WO 04/058266, WO 04/064778, and WO 04/069162 disclose diverse β-amino acid based phenylbutanamide derivatives described as DPP IV inhibitors. WO 04/083212, WO 04/085661, WO 04/087650 and WO 04/085378 disclose the processes for the preparation of enantiomerically enriched beta amino acid derivatives said to be useful for the asymmetric synthesis of biological active molecules. WO 98/17273 discloses use of butyric acid derivatives said to protect against hair loss or damage in human cancer patients undergoing chemo- or radiation therapy. WO 96/26183 discloses 1-aryl-2-aylamino-ethane compounds and their use as neurokinin 1-antagonist. U.S. Pat. No. 5,665,876 discloses 3-(aminoacyl-amino) saccharides, which have been said to clarify the biological function of glycoproteins. WO 05/040095 and WO 05/056003 disclose compounds described as having dipeptidyl peptidase-IV inhibitory activity. The patent applications WO 01/055105, WO 03/000180, WO 05/056103, WO 04/050022, WO 04/043490, WO 04/089362, WO 04/103276, and WO 05/095343 describe the β-amino acid based derivatives as the inhibitors of DPP IV.

### SUMMARY OF THE INVENTION

The present invention provides compounds containing β-amino acid derivatives possessing dipeptidyl peptidase-IV enzyme inhibitory activity. Also provided are processes for synthesizing such compounds.

These compounds can be used in treatment of conditions mediated by DPP IV, such as diabetes, especially, type 2 diabetes mellitus as well as pre-diabetes, diabetic dyslipidemia, metabolic acidosis, ketosis, satiety disorders, and obesity. These inhibitors can also be used for treating conditions manifested by a variety of metabolic, neurological, anti-inflammatory, and autoimmune disorders like inflammatory disease, multiple sclerosis, rheumatoid arthritis viral, cancer and gastrointestinal disorders. The compounds of this invention can also be used for treatment of infertility arising due to polycystic ovary syndrome.

Pharmaceutical compositions containing such compounds are provided together with the pharmaceutically acceptable carriers or diluents, which can be used for the treatment of dipeptidyl peptidase-IV mediated pathologies. These pharmaceutical compositions may be administered or coadministered by a wide variety of routes, for example, oral or parenteral. The composition may also be administered or coadministered in slow release dosage forms.

Although, one specific enantiomer has been shown by way of example, the racemates, diastereomers, N-oxides, polymorphs, pharmaceutically acceptable salts and pharmaceutically acceptable solvates of these compounds, prodrugs and metabolites, having the same type of activity, are also provided as well as pharmaceutical compositions comprising the compounds, their metabolites, racemates, enantiomers, N-oxides, polymorphs, solvates, prodrugs or pharmaceutically acceptable salts thereof, in combination with a pharmaceutically acceptable carrier and optionally included excipients.

Other objects will be set forth in accompanying description and in the part will be apparent from the description or may be learnt by the practice of the invention.

In accordance with one aspect of the invention, are provided compounds having the structure of formula I

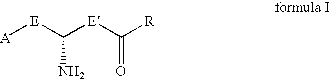

including pharmaceutically acceptable salts, pharmaceutically acceptable solvates, enantiomers, diastereomers, polymorphs, prodrugs, metabolites or N-oxides thereof,
wherein
A is selected from aryl or heteroaryl group.
E and E′ are independently —(CRₐR_{b})₁— (wherein 1 is an integer of 1 to 2 and Rₐ and R_{b} are independently selected from the group consisting of hydrogen, alkyl, alkenyl, alkynyl, cycloalkyl, aryl, heteroaryl or heterocyclyl; Rₐ and R_{b} can together form a ring, which can be optionally unsaturated); and
R can be selected from the groups a to c:

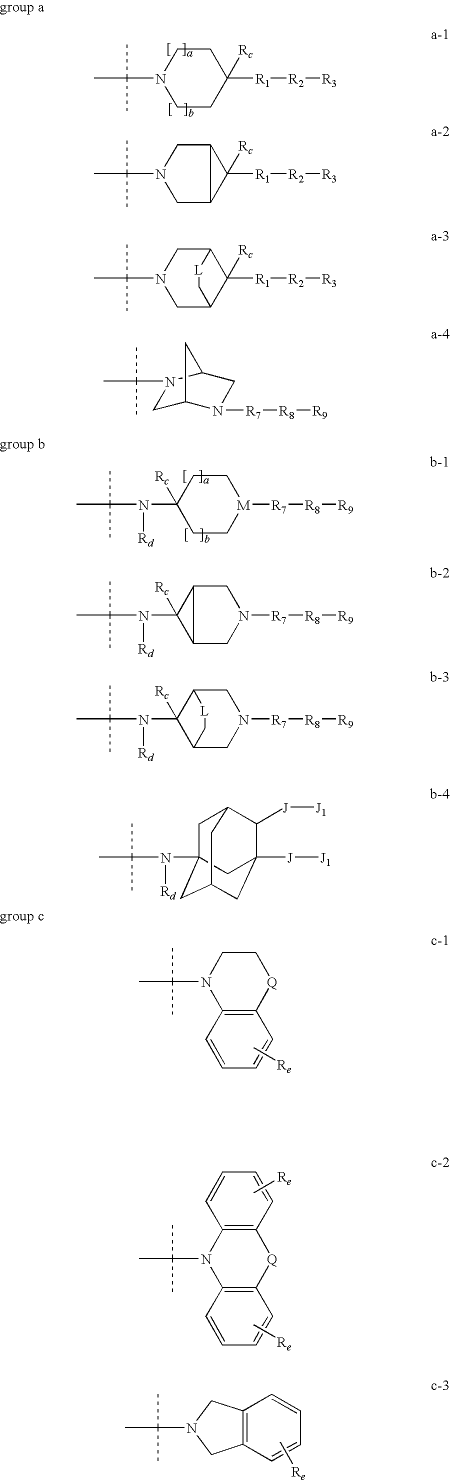

wherein
R_{c} is hydrogen or alkyl;
R_{d} is hydrogen, alkyl, aryl, heteroaryl, heterocyclyl or cycloalkyl;
Rₑ is hydrogen, alkyl, halogen, cyano, carboxy, hydroxyl, alkoxy, carbonyl or amino;
a and b are an integer of 0-2;
J is a bond, —O—, —NR_{f}—, —NR_{f}CO—, —NR_{f}CONR_{f}—, —NR_{f}SO₂—, —NR_{f}C(O)O—, or —OCONR_{f}—, wherein R_{f} refers to hydrogen, alkyl, cycloalkyl, aryl, heteroaryl or heterocyclyl;
J₁ is hydrogen, alkyl, cycloalkyl, aryl, heteroaryl or heterocyclyl (when J is —NR_{f}SO₂—, or NR_{f}C(O)O—, then J₁ is not hydrogen);
L is (CH₂)ₚ wherein p is an integer of 1-2;

### M is CH or N;

Q is (CH₂)_{q}, O or S(O)_{q} wherein q is an integer of 0-2;
R₁ is —(CRₐR_{b})ₘ— wherein m is an integer of 0-1;
R₂ is —NR_{f}—, —O—, —CO—, —CS—, —CONR_{f}—, —NR_{f}CO—, —NR_{f}CONR_{f}—, —NR_{f}SO₂—, —NR_{f}COO—, or
—OCONR_{f}—; wherein R_{f} is defined as above;
R₃ is alkyl, alkenyl, alkynyl, cycloalkyl, aryl, heteroaryl or heterocyclyl;
R₇ is no atom, —CO—, —CS—, and —SO₂—;
R₈ is no atom, —O— or —NR_{f}—; and
R₉ is alkyl, alkenyl, alkynyl, cycloalkyl, aryl, heteroaryl or heterocyclyl, with the following provisos:
(i) when A, E, and E′ are defined as earlier, R as a-1 and a-2 (group a), R₁ as —(CRₐR_{b})ₘ-{wherein m=0 or m=1 when a=b=1}, and R₂ as —NR_{f}—, then R₃ cannot be a heteroaryl,
(ii) when A, E, and E′ are defined as earlier, R as a-1 (group a) {wherein a is an integer of 0 and b is an integer of 1}, R₁ as —(CRₐR_{b})ₘ-{wherein m is an integer of 0}, and R₃ as defined earlier, then R₂ cannot be —CONR_{f}.
(iii) when A, E, and E′ are defined as earlier, R as a-1 (group a) wherein a is an integer of 0 and b is an integer of 0-2, R₂ as —O— and —NR_{f}— and R₃ as defined earlier, then R₁ cannot be —(CRₐR_{b})ₘ— [wherein m is an integer of 1].
(iv) when A, E, and E′ are defined as earlier, R as b-1 (when M is N) and b-2 (group b) or a-4 (group a) wherein R₇ and R₈ are no atom, then R₉ cannot be a heteroaryl,
(v) when A, E, and E′ are defined as earlier, R as a-4 (group a), b-1 (when M is N), b-2, and b-3, (group b), and R₇ as no atom, then R₈ cannot be —O— or —NR_{f}—.

In yet other embodiment, compounds are provided that include, for example,
Compound No. 1: (3R)-3-Amino-N-[1-(morpholin-4-ylcarbonyl)piperidin-4-yl]-4-(2,4,5-trifluorophenyl)butanamide and its trifluoroacetic acid salt,
Compound No. 2: (3R)-3-Amino-N-{1-[(4-fluorophenyl)sulphonyl]piperidin-4-yl}-4-(2,4,5-trifluorophenyl)butanamide and its 4-methylbenzenesulfonic acid salt,
Compound No. 3: (3R)-3-Amino-N-[1-(4-fluorobenzoyl)piperidin-4-yl]-4-(2,4,5-trifluoro phenyl)butanamide and its 4-methylbenzenesulfonic acid salt,
Compound No. 4: N-{1-[(3R)-3-Amino-4-(2,4,5-trifluorophenyl)butanoyl]piperidin-4-yl}-4-fluorobenzamide and its trifluoroacetic acid salt,
Compound No. 5: (3R)-3-Amino-N-[1-(methylsulphonyl)piperidin-4-yl]-4-(2,4,5-trifluoro phenyl)butanamide and its 4-methylbenzenesulfonic acid salt,
Compound No. 6: (3R)-3-Amino-N-(3-hydroxy-1-adamantyl)-4-(2,4,5-trifluorophenyl) butanamide and its 4-methylbenzenesulfonic acid salt,
Compound No. 7: 4-{[(3R)-3-Amino-4-(2,4,5-trifluorophenyl)butanoyl]amino}-N-(4-chloro phenyl)piperidine-1-carboxamide and its hydrochloride salt,
Compound No. 8: (3R)-3-Amino-N-[(1R,5S)-3-(2-thienylsulphonyl)-3-azabicyclo[3.1.0]hex-6-yl]-4-(2,4,5-trifluorophenyl)butanamide and its trifluoroacetic acid salt,
Compound No. 9: (3R)-3-Amino-N-[(1R,5S)-3-(4-cyanobenzoyl)-3-azabicyclo[3.1.0]hex-6-yl]-4-(2,4,5-trifluorophenyl)butanamide and its trifluoroacetic acid salt,
Compound No. 10: (3R)-3-Amino-N-[(1R,5S)-3-(2,6-difluorobenzoyl)-3-azabicyclo[3.1.0]hex-6-yl]-4-(2,4,5-trifluorophenyl)butanamide and its trifluoroacetic acid salt,
Compound No. 11: N-{1-[(3R)-3-Amino-4-(2,4,5-trifluorophenyl)butanoyl]piperidin-4-yl}-4-fluorobenzenesulfonamide and its trifluoroacetic acid salt,
Compound No. 12: N-{1-[(3R)-3-Amino-4-(2,4,5-trifluorophenyl)butanoyl]piperidin-4-yl}morpholine-4-carboxamide and its trifluoroacetic acid salt,
Compound No. 13: 1-{1-[(3R)-3-Amino-4-(2,4,5-trifluorophenyl)butanoyl]piperidin-4-yl}-3-(4-chlorophenyl)urea and its trifluoroacetic acid salt,
Compound No. 14: N-{1-[(3R)-3-Amino-4-(2,4,5-trifluorophenyl)butanoyl]piperidin-4-yl}-3-fluoro-4-methoxybenzamide and its trifluoroacetic acid salt,
Compound No. 15: N-{1-[(3R)-3-Amino-4-(2,4,5-trifluorophenyl)butanoyl]piperidin-4-yl}-2-propanesulfonamide and its trifluoroacetic acid salt,
Compound No. 16: (3R)-3-Amino-N-[(1R,5S)-3-(4-trifluorobenzenesulphonyl)-3-azabicyclo [3.1.0]hex-6-yl]-4-(2,4,5-trifluorophenyl)butanamide and its trifluoroacetic acid salt,
Compound No. 17: (3R)-3-Amino-N-[(1R,5S)-3-(thiophene-2-carbonyl)-3-azabicyclo[3.1.0]hex-6-yl]-4-(2,4,5-trifluorophenyl)butanamide and its trifluoroacetic acid salt,
Compound No. 18: (3R)-3-Amino-N-[(1R,5S)-3-(ethanesulphonyl)-3-azabicyclo[3.1.0]hex-6-yl]-4-(2,4,5-trifluorophenyl)butanamide and its trifluoroacetic acid salt,
Compound No. 19: (3R)-3-Amino-N-[(1R,5S)-3-(4-methylbenznesulphonyl)-3-azabicyclo[3.1.0] hex-6-yl]-4-(2,4,5-trifluorophenyl)butanamide and its trifluoroacetic acid salt,
Compound No. 20: 4-[({(3R)-1-[(3R)-3-Amino-4-(2,4,5-trifluorophenyl)butanoyl]pyrrolidin-3-yl}oxy)methyl]benzonitrile and its trifluoroacetic acid salt,
Compound No. 21: (2R)-4-{(3S)-3-[(4-Fluorobenzyl)oxy]pyrrolidin-1-yl}-4-oxo-1-(2,4,5-trifluorophenyl)butan-2-amine and its trifluoroacetic acid salt,
Compound No. 22: 4-[({(3S)-1-[(3R)-3-Amino-4-(2,4,5-trifluorophenyl)butanoyl]pyrrolidin-3-yl}oxy)methyl]benzonitrile and its trifluoroacetic acid salt,
Compound No. 23: N-{1-[(3R)-3-amino-4-(2,4,5-trifluorophenyl)butanoyl]piperidin-4-yl}-2,2,2-trifluoroethanesulfonamide and its trifluoroacetic acid salt,
Compound No. 24: N-{1-[(3R)-3-amino-4-(2,4,5-trifluorophenyl)butanoyl]piperidin-4-yl}-4-cyanobenzenesulfonamide and its trifluoroacetic acid salt,
Compound No. 25: N-{1-[3-amino-4-(2,4,5-trifluorophenyl)butanoyl]piperidin-4-yl}-2,4-difluorobenzenesulfonamide and its trifluoroacetic acid salt,
Compound No. 26: Methyl 5-[({1-[(3R)-3-amino-4-(2,4,5-trifluorophenyl)butanoyl]piperidin-4-yl} amino)sulfonyl]-4-methylthiophene-2-carboxylate,
Compound No. 27: N-{1-[(3R)-3-amino-4-(2,4,5-trifluorophenyl)butanoyl]piperidin-4-yl}-1,3,5-trimethyl-1H-pyrazole-4-sulfonamide,
Compound No. 28: methyl 4-[({1-[(3R)-3-amino-4-(2,4,5-trifluorophenyl)butanoyl]piperidin-4-yl} amino)sulfonyl]-2,5-dimethyl-3-furoate,
Compound No. 29: N-{1-[(3R)-3-amino-4-(2,4,5-trifluorophenyl)butanoyl]piperidin-4-yl}-4-fluorobenzenesulfonamide,
Compound No. 30: 4-acetyl-N-{1-[(3R)-3-amino-4-(2,4,5-trifluorophenyl)butanoyl] piperidin-4-yl}benzenesulfonamide and its trifluoroacetic acid salt,
Compound No. 31: N-{1-[(3R)-3-amino-4-(2,4,5-trifluorophenyl)butanoyl]piperidin-4-yl}-2,4-difluorobenzenesulfonamide and its trifluoroacetic acid salt,
Compound No. 32: N-{1-[(3R)-3-amino-4-(2,4,5-trifluorophenyl)butanoyl]piperidin-4-yl} methane sulfonamide and its trifluoroacetic acid salt,
Compound No. 33: methyl 5-[({1-[(3R)-3-amino-4-(2,4,5-trifluorophenyl)butanoyl] piperidin-4-yl} amino) sulfonyl]-4-methylthiophene-2-carboxylate and its trifluoroacetic acid salt,
Compound No. 34: N-{1-[(3R)-3-amino-4-(2,4,5-trifluorophenyl)butanoyl]piperidin-4-yl} propane-2-sulfonamide and its trifluoroacetic acid salt,
Compound No. 35: N-{(3S)-1-[(3R)-3-amino-4-(2,4,5-trifluorophenyl)butanoyl]piperidin-3-yl}ethane sulfonamide and its trifluoroacetic acid salt,
Compound No. 36: 4-(1,3-dihydro-2H-isoindol-2-yl)-4-oxo-1-(2,4,5-trifluorophenyl)butan-2-amine and its trifluoroacetic acid salt,
Compound No. 37: N-{1-[3-amino-4-(2,4,5-trifluorophenyl)butanoyl]piperidin-4-yl}propanamide and its trifluoroacetic acid salt,
Compound No. 38: N-{1-[(3R)-3-amino-4-(2,4,5-trifluorophenyl)butanoyl]piperidin-4-yl}acetamide and its trifluoroacetic acid salt,
Compound No. 39: N-{1-[(3R)-3-amino-4-(2,4,5-trifluorophenyl)butanoyl]piperidin-4-yl}-4-fluorobenzamide,
Compound No. 40: 2-({1-[(3R)-3-amino-4-(2,4,5-trifluorophenyl)butanoyl]piperidin-4-yl} amino)-4,6-difluorobenzonitrile and its trifluoroacetic acid salt,
Compound No. 41: 2-({1-[(3R)-3-amino-4-(2,4,5-trifluorophenyl)butanoyl]piperidin-4-yl} amino)benzonitrile and its trifluoroacetic acid salt,
Compound No. 42: 4-({1-[(3R)-3-amino-4-(2,4,5-trifluorophenyl)butanoyl]piperidin-4-yl} amino)-2,6-difluorobenzonitrile and its 4-methylbenzenesulfonic acid salt,
Compound No. 43: 4-({1-[(3R)-3-amino-4-(2,4,5-trifluorophenyl)butanoyl]piperidin-4-yl} amino)-2,6-difluorobenzonitrile and its trifluoroacetic acid salt,
Compound No. 44: 2-({1-[(3R)-3-amino-4-(2,4,5-trifluorophenyl)butanoyl]piperidin-4-yl} amino)benzonitrile and its trifluoroacetic acid salt,
Compound No. 45: 4-({1-[(3R)-3-amino-4-(2,4,5-trifluorophenyl)butanoyl]piperidin-4-yl} amino)benzonitrile and its trifluoroacetic acid salt,
Compound No. 46: 4-({1-[(3R)-3-amino-4-(2,4,5-trifluorophenyl)butanoyl]piperidin-4-yl} amino)benzonitrile and its trifluoroacetic acid salt,
Compound No. 47: 4-({1-[(3R)-3-amino-4-(2,4,5-trifluorophenyl)butanoyl]piperidin-4-yl} amino)-2-(trifluoromethyl)benzonitrile and its trifluoroacetic acid salt,
Compound No. 48: Ethyl ({(3S)-1-[(3R)-3-amino-4-(2,4,5-trifluorophenyl)butanoyl]pyrrolidin-3-yl}oxy)acetate and its trifluoroacetic acid salt,
Compound No. 49: (2R)-4-oxo-4-[5-(2-thienylacetyl)-2,5-diazabicyclo[2.2.1]hept-2-yl]-1-(2,4,5-trifluorophenyl)butan-2-amine and its trifluoroacetic acid salt,
Compound No. 50: (2R)-4-oxo-4-[5-(trifluoroacetyl)-2,5-diazabicyclo[2.2.1]hept-2-yl]-1-(2,4,5-trifluorophenyl)butan-2-amine and its hydrochloride salt,
Compound No. 51: (2R)-4-oxo-4-(5-propionyl-2,5-diazabicyclo[2.2.1]hept-2-yl)-1-(2,4,5-trifluoro phenyl) butan-2-amine and its hydrochloride salt,
Compound No. 52: 5-[(3R)-3-amino-4-(2,4,5-trifluorophenyl)butanoyl]-N-(4-cyanophenyl)-2,5-diazabicyclo[2.2.1]heptane-2-carboxamide and its 4-methylbenzenesulfonic acid salt,
Compound No. 53: (2R)-4-(5-acetyl-2,5-diazabicyclo[2.2.1]hept-2-yl)-4-oxo-1-(2,4,5-trifluoro-phenyl)butan-2-amine and its trifluoroacetic acid salt,
Compound No. 54: (1S,4S)-5-[(3R)-3-amino-4-(2,4,5-trifluorophenyl)butanoyl]-N-(4-fluoro-phenyl)-2,5-diazabicyclo[2.2.1]heptane-2-carboxamide and its trifluoroacetic acid salt,
Compound No. 55: (1S,4S)-5-[(3R)-3-amino-4-(2,4,5-trifluorophenyl)butanoyl]-N-(4-methoxy-phenyl)-2,5-diazabicyclo[2.2.1]heptane-2-carboxamide and its trifluoroacetic acid salt,
Compound No. 56: (1S,4S)-5-[(3R)-3-amino-4-(2,4,5-trifluorophenyl)butanoyl]-N-[4-(trifluoro-methyl)phenyl]-2,5-diazabicyclo[2.2.1]heptane-2-carboxamide and its trifluoroacetic acid salt,
Compound No. 57: (1S,4S)-5-[(3R)-3-amino-4-(2,4,5-trifluorophenyl)butanoyl]-N-benzyl-2,5-diazabicyclo[2.2.1]heptane-2-carboxamide and its trifluoroacetic acid salt,
Compound No. 58: (1S,4S)-5-[(3R)-3-amino-4-(2,4,5-trifluorophenyl)butanoyl]-N-(4-methyl-phenyl)-2,5-diazabicyclo[2.2.1]heptane-2-carboxamide and its trifluoroacetic acid salt,
Compound No. 59: (1S,4S)-5-[(3R)-3-amino-4-(2,4,5-trifluorophenyl)butanoyl]-N-tert-butyl-2,5-diaza-bicyclo[2.2.1]heptane-2-carboxamide and its trifluoroacetic acid salt,
Compound No. 60: (2R)-4-{5-[(3-fluorophenyl)sulfonyl]-2,5-diazabicyclo[2.2.1]hept-2-yl}-4-oxo-1-(2,4,5-trifluorophenyl)butan-2-amine and its trifluoroacetic acid salt,
Compound No. 61: (1S,4S)-5-[(3R)-3-amino-4-(2,4,5-trifluorophenyl)butanoyl]-N-(3-fluoro-phenyl)-2,5-diazabicyclo[2.2.1]heptane-2-carboxamide and its trifluoroacetic acid salt,
Compound No. 62: (1S,4S)-5-[(3R)-3-amino-4-(2,4,5-trifluorophenyl)butanoyl]-N-[2-(trifluoromethyl)phenyl]-2,5-diazabicyclo[2.2.1]heptane-2-carboxamide and its trifluoroacetic acid salt,
Compound No. 63: (1S,4S)-5-[(3R)-3-amino-4-(2,4,5-trifluorophenyl)butanoyl]-N-(2-methyl-phenyl)-2,5-diazabicyclo[2.2.1]heptane-2-carboxamide and its trifluoroacetic acid salt,
Compound No. 64: (1S,4S)-5-[(3R)-3-amino-4-(2,4,5-trifluorophenyl)butanoyl]-N-(4-nitro-phenyl)-2,5-diazabicyclo[2.2.1]heptane-2-carboxamide and its trifluoroacetic acid salt,
Compound No. 65: 4-{(1S,4S)-5-[(3R)-3-amino-4-(2,4,5-trifluorophenyl)butanoyl]-2,5-diaza-bicyclo[2.2.1]hept-2-yl}-2-chlorobenzonitrile and its trifluoroacetic acid salt,
Compound No. 66: 2-{(1S,4S)-5-[(3R)-3-amino-4-(2,4,5-trifluorophenyl)butanoyl]-2,5-diaza-bicyclo[2.2.1]hept-2-yl}-6-fluorobenzonitrile and its trifluoroacetic acid salt,
Compound No. 67: 4-{(1S,4S)-5-[(3R)-3-amino-4-(2,4,5-trifluorophenyl)butanoyl]-2,5-diaza-bicyclo[2.2.1]hept-2-yl}-3-fluorobenzonitrile and its trifluoroacetic acid salt,
Compound No. 68: (1S,4S)-5-[(3R)-3-amino-4-(2,4,5-trifluorophenyl)butanoyl]-N-cyclohexyl-2,5-diazabicyclo[2.2.1]heptane-2-carboxamide and its trifluoroacetic acid salt,
Compound No. 69: (1S,4S)-5-[(3R)-3-amino-4-(2,4,5-trifluorophenyl)butanoyl]-N-(3-methoxy-phenyl)-2,5-diazabicyclo[2.2.1]heptane-2-carboxamide and its trifluoroacetic acid salt,
Compound No. 70: (1S,4S)-5-[(3R)-3-amino-4-(2,4,5-trifluorophenyl) butanoyl]-N-(2-fluoro-phenyl)-2,5-diazabicyclo[2.2.1]heptane-2-carboxamide and its trifluoroacetic acid salt,
Compound No. 71: (2R)-4-[5-(ethylsulfonyl)-2,5-diazabicyclo[2.2.1]hept-2-yl]-4-oxo-1-(2,4,5-trifluorophenyl)butan-2-amine and its trifluoroacetic acid salt,
Compound No. 72: (1S,4S)-5-[(3R)-3-amino-4-(2,4,5-trifluorophenyl)butanoyl]-N-(2,4-dimethoxyphenyl)-2,5-diazabicyclo[2.2.1]heptane-2-carboxamide and its trifluoroacetic acid salt,
Compound No. 73: (1S,4S)-5-[(3R)-3-amino-4-(2,4,5-trifluorophenyl)butanoyl]-N-isopropyl-2,5-diazabicyclo[2.2.1]heptane-2-carboxamide and its trifluoroacetic acid salt,
Compound No. 74: 4-{5-[(3R)-3-amino-4-(2,4,5-trifluorophenyl)butanoyl]-2,5-diazabicyclo-[2.2.1]hept-2-yl}-2-(trifluoromethyl)benzonitrile and its trifluoroacetic acid salt,
Compound No. 75: (1S,4S)-5-[(3R)-3-amino-4-(2,4,5-trifluorophenyl)butanoyl]-N-[4-(benzyl-oxy)phenyl]-2,5-diazabicyclo[2.2.1]heptane-2-carboxamide and its trifluoroacetic acid salt,
Compound No. 76: (2R)-4-[5-(4-fluorobenzoyl)-2,5-diazabicyclo[2.2.1]hept-2-yl]-4-oxo-1-(2,4,5-trifluorophenyl)butan-2-amine and its trifluoroacetic acid salt,
Compound No. 77: (1S,4S)-5-[(3R)-3-amino-4-(2,4,5-trifluorophenyl)butanoyl]-N-(3,4,5-tri-methoxyphenyl)-2,5-diazabicyclo[2.2.1]heptane-2-carboxamide and its trifluoroacetic acid salt,
Compound No. 78: 5-[3-amino-4-(2,4,5-trifluorophenyl)butanoyl]-N-(4-fluorophenyl)-2,5-diaza-bicyclo[2.2.1]heptane-2-carboxamide and its trifluoroacetic acid salt,
Compound No. 79: (1S,4S)-5-[3-amino-4-(2,4,5-trifluorophenyl)butanoyl]-N-(2,6-diisopropyl phenyl)-2,5-diazabicyclo[2.2.1]heptane-2-carboxamide and its trifluoroacetic acid salt,
Compound No. 80: methyl 2-[({(1S,4S)-5-[(3R)-3-amino-4-(2,4,5-trifluorophenyl)butanoyl]-2,5-diaza-bicyclo[2.2.1]hept-2-yl}carbonyl)amino]benzoate and its trifluoroacetic acid salt,
Compound No. 81: (2R)-4-oxo-4-[5-(2-thienylsulfonyl)-2,5-diaza-bicyclo[2.2.1]hept-2-yl]-1-(2,4,5-trifluorophenyl)butan-2-amine and its trifluoroacetic acid salt,
Compound No. 82: (1S,4S)-5-[(3R)-3-amino-4-(2,4,5-trifluorophenyl)butanoyl]-N-(5-chloro-2-methoxy phenyl)-2,5-diazabicyclo [2.2.1]heptane-2-carboxamide and its trifluoroacetic acid salt,
Compound No. 83: 4-({5-[(3R)-3-amino-4-(2,4,5-trifluoro phenyl)butanoyl]-2,5-diazabicyclo [2.2.1]hept-2-yl}sulfonyl)benzonitrile and its trifluoroacetic acid salt,
Compound No. 84: (1S,4S)-5-[(3R)-3-amino-4-(2,4,5-trifluorophenyl)butanoyl]-N-(2,3-dichlorophenyl)-2,5-diazabicyclo [2.2.1]heptane-2-carboxamide and its trifluoroacetic acid salt,
Compound No. 85: (2R)-4-{(1S,4S)-5-[(3,5-difluorophenyl) sulfonyl]-2,5-diazabicyclo [2.2.1]hept-2-yl}-4-oxo-1-(2,4,5-trifluorophenyl) butan-2-amine and its trifluoroacetic acid salt,
Compound No. 86: (1S,4S)—N-(4-acetylphenyl)-5-[(3R)-3-amino-4-(2,4,5-trifluorophenyl) butanoyl]-2,5-diazabicyclo[2.2.1]heptane-2-carboxamide and its trifluoroacetic acid salt,
Compound No. 87: methyl 5-[(3R)-3-amino-4-(2,4,5-trifluorophenyl)butanoyl]-2,5-diazabicyclo [2.2.1]heptane-2-carboxylate and its trifluoroacetic acid salt,
Compound No. 88: (1S,4S)-5-[(3R)-3-amino-4-(2,4,5-trifluorophenyl)butanoyl]-N-(2,5-dimethoxyphenyl)-2,5-diazabicyclo[2.2.1]heptane-2-carboxamide and its trifluoroacetic acid salt,
Compound No. 89: ethyl 5-[(3R)-3-amino-4-(2,4,5-trifluorophenyl)butanoyl]-2,5-diazabicyclo [2.2.1]heptane-2-carboxylate and its trifluoroacetic acid salt,
Compound No. 90: (2R)-4-oxo-4-[5-(propylsulfonyl)-2,5-diazabicyclo[2.2.1]hept-2-yl]-1-(2,4,5-trifluoro-phenyl)butan-2-amine and its trifluoroacetic acid salt,
Compound No. 91: (2R)-4-[5-(isopropylsulfonyl)-2,5-diazabicyclo[2.2.1]hept-2-yl]-4-oxo-1-(2,4,5-trifluoro phenyl)butan-2-amine and its trifluoroacetic acid salt,
Compound No. 92: (2R)-4-[5-(butylsulfonyl)-2,5-diazabicyclo[2.2.1]hept-2-yl]-4-oxo-1-(2,4,5-trifluoro-phenyl)butan-2-amine and its trifluoroacetic acid salt,
Compound No. 93: (2R)-4-oxo-4-[5-(phenylsulfonyl)-2,5-diazabicyclo[2.2.1]hept-2-yl]-1-(2,4,5-trifluorophenyl)butan-2-amine and its trifluoroacetic acid salt,
Compound No. 94: (2R)-4-[5-(morpholin-4-ylcarbonyl)-2,5-diazabicyclo[2.2.1]hept-2-yl]-4-oxo-1-(2,4,5-trifluorophenyl)butan-2-amine and its trifluoroacetic acid salt,
Compound No. 95: (2R)-4-oxo-4-(5-{[3-(trifluoromethoxy)phenyl]sulfonyl}-2,5-diazabicyclo [2.2.1]hept-2-yl)-1-(2,4,5-trifluorophenyl)butan-2-amine and its trifluoroacetic acid salt,
Compound No. 96: (2R)-4-[5-(methylsulfonyl)-2,5-diazabicyclo[2.2.1]hept-2-yl]-4-oxo-1-(2,4,5-trifluorophenyl)butan-2-amine and its trifluoroacetic acid salt,
Compound No. 97: (1S,4S)-5-[(3R)-3-amino-4-(2,4,5-trifluorophenyl)butanoyl]-N-(2,6-difluoro-phenyl)-2,5-diazabicyclo [2.2.1]heptane-2-carboxamide and its trifluoroacetic acid salt,
Compound No. 98: (1S,4S)-5-[(3R)-3-amino-4-(2,4,5-trifluorophenyl)butanoyl]-N-(2,4,6-trifluoro-phenyl)-2,5-diazabicyclo[2.2.1]heptane-2-carboxamide and its trifluoroacetic acid salt,
Compound No. 99: (1S,4S)—N-(3-acetylphenyl)-5-[(3R)-3-amino-4-(2,4,5-trifluorophenyl) butanoyl]-2,5-diazabicyclo[2.2.1]heptane-2-carboxamide and its trifluoroacetic acid salt,
Compound No. 100: (1S,4S)-5-[(3R)-3-amino-4-(2,4,5-trifluorophenyl)butanoyl]-N-(2,5-dichloro-phenyl)-2,5-diazabicyclo[2.2.1]heptane-2-carboxamide and its trifluoroacetic acid salt,
Compound No. 101: (1S,4S)-5-[(3R)-3-amino-4-(2,4,5-trifluorophenyl)butanoyl]-N-(4-isopropyl-phenyl)-2,5-diazabicyclo[2.2.1]heptane-2-carboxamide and its trifluoroacetic acid salt,
Compound No. 102: (1S,4S)-5-[(3R)-3-amino-4-(2,4,5-trifluorophenyl)butanoyl]-N-(4-butyl-phenyl)-2,5-diazabicyclo[2.2.1]heptane-2-carboxamide and its trifluoroacetic acid salt,
Compound No. 103: (1S,4S)-5-[(3R)-3-amino-4-(2,4,5-trifluorophenyl)butanoyl]-N-(4-ethoxy-phenyl)-2,5-diazabicyclo[2.2.1]heptane-2-carboxamide and its trifluoroacetic acid salt,
Compound No. 104: (1S,4S)-5-[(3R)-3-amino-4-(2,4,5-trifluorophenyl)butanoyl]-N-(3-ethyl-phenyl)-2,5-diazabicyclo[2.2.1]heptane-2-carboxamide and its trifluoroacetic acid salt,
Compound No. 105: (1S,4S)-5-[(3R)-3-amino-4-(2,4,5-trifluorophenyl)butanoyl]-N-(2-isopropyl-6-methylphenyl)-2,5-diazabicyclo[2.2.1]heptane-2-carboxamide and its trifluoroacetic acid salt,
Compound No. 106: (1S,4S)-5-[(3R)-3-amino-4-(2,4,5-trifluorophenyl)butanoyl]-N-mesityl-2,5-diazabicyclo[2.2.1]heptane-2-carboxamide and its trifluoroacetic acid salt,
Compound No. 107: (1S,4S)-5-[(3R)-3-amino-4-(2,4,5-trifluorophenyl)butanoyl]-N-(4-methoxy-2-methylphenyl)-2,5-diazabicyclo[2.2.1]heptane-2-carboxamide and its trifluoroacetic acid salt,
Compound No. 108: (1S,4S)-5-[(3R)-3-amino-4-(2,4,5-trifluorophenyl)butanoyl]-N-(4-phenoxy-phenyl)-2,5-diazabicyclo[2.2.1]heptane-2-carboxamide and its trifluoroacetic acid salt,
Compound No. 109: (2R)-4-[(1R,4R)-5-(cyclohexylcarbonyl)-2,5-diazabicyclo[2.2.1]hept-2-yl]-4-oxo-1-(2,4,5-trifluorophenyl) butan-2-amine and its trifluoroacetic acid salt,
Compound No. 110: (2R)-4-[(1R,4R)-5-(cyclopropyl carbonyl)-2,5-diazabicyclo [2.2.1]hept-2-yl]-4-oxo-1-(2,4,5-trifluorophenyl) butan-2-amine and its trifluoroacetic acid salt,
Compound No. 111: (2R)-4-[(1R,4R)-5-(3,5-difluorobenzyl)-2,5-diazabicyclo[2.2.1]hept-2-yl]-4-oxo-1-(2,4,5-trifluorophenyl)butan-2-amine and its trifluoroacetic acid salt,
Compound No. 112: (1R,4R)-5-[(3R)-3-amino-4-(2,4,5-trifluorophenyl)butanoyl]-N-(2-ethoxyphenyl)-2,5-diazabicyclo[2.2.1]heptane-2-carboxamide and its trifluoroacetic acid salt,
Compound No. 113: (1R,4R)-5-[(3R)-3-amino-4-(2,4,5-trifluorophenyl)butanoyl]-N-(2-ethylphenyl)-2,5-diazabicyclo[2.2.1]heptane-2-carboxamide and its trifluoroacetic acid salt,
Compound No. 114: methyl 3-[({(1R,4R)-5-[(3R)-3-amino-4-(2,4,5-trifluorophenyl)butanoyl]-2,5-diazabicyclo[2.2.1]hept-2-yl}carbonyl)amino]benzoate and its trifluoroacetic acid salt,
Compound No. 115: (1R,4R)-5-[(3R)-3-amino-4-(2,4,5-trifluorophenyl)butanoyl]-N-(4-ethylphenyl)-2,5-diazabicyclo[2.2.1]heptane-2-carboxamide and its trifluoroacetic acid salt,
Compound No. 116: (1R,4R)-5-[(3R)-3-amino-4-(2,4,5-trifluorophenyl)butanoyl]-N-(3-chloro-4-methylphenyl)-2,5-diazabicyclo[2.2.1]heptane-2-carboxamide and its trifluoroacetic acid salt,
Compound No. 117: (1R,4R)-5-[(3R)-3-amino-4-(2,4,5-trifluorophenyl)butanoyl]-N-[3-(methylthio)phenyl]-2,5-diazabicyclo[2.2.1]heptane-2-carboxamide and its trifluoroacetic acid salt,
Compound No. 118: (1R,4R)-5-[(3R)-3-amino-4-(2,4,5-trifluorophenyl)butanoyl]-N-(2,4-difluorophenyl)-2,5-diazabicyclo[2.2.1]heptane-2-carboxamide and its trifluoroacetic acid salt,
Compound No. 119: (1R,4R)-5-[(3R)-3-amino-4-(2,4,5-trifluorophenyl)butanoyl]-N-(2,4-dimethylphenyl)-2,5-diazabicyclo[2.2.1]heptane-2-carboxamide and its trifluoroacetic acid salt,
Compound No. 120: (1R,4R)-5-[(3R)-3-amino-4-(2,4,5-trifluorophenyl)butanoyl]-N-(3,4-dichlorophenyl)-2,5-diazabicyclo[2.2.1]heptane-2-carboxamide and its trifluoroacetic acid salt,
Compound No. 121: (1R,4R)-5-[(3R)-3-amino-4-(2,4,5-trifluorophenyl)butanoyl]-N-[4-chloro-3-(trifluoromethyl)phenyl]-2,5-diazabicyclo[2.2.1]heptane-2-carboxamide and its trifluoroacetic acid salt,
Compound No. 122: (1R,4R)-5-[(3R)-3-amino-4-(2,4,5-trifluorophenyl)butanoyl]-N-(3-cyanophenyl)-2,5-diazabicyclo[2.2.1]heptane-2-carboxamide and its trifluoroacetic acid salt,
Compound No. 123: (1R,4R)-5-[(3R)-3-amino-4-(2,4,5-trifluorophenyl)butanoyl]-N-(2-isopropylphenyl)-2,5-diazabicyclo[2.2.1]heptane-2-carboxamide and its trifluoroacetic acid salt, and
Compound No. 124: (1R,4R)-5-[(3R)-3-amino-4-(2,4,5-trifluorophenyl)butanoyl]-N-[3,5-bis(trifluoromethyl)phenyl]-2,5-diazabicyclo[2.2.1]heptane-2-carboxamide and its trifluoroacetic acid salt.

In yet another embodiment, the present invention relates to the therapeutically effective dose of a compound of formula I in combination with one or more of other therapeutic agents used for treating metabolic disorders. The examples of such therapeutic agents include, but not limited to,
1) antihyperglycaemic agents: (a) insulin sensitizers, (i) PPAR agonists, for example, PPARγ agonists (e.g., rosiglitazone and pioglitazone), PPARα/γ dual agonists (e.g., tesaglitazar and muraglitazar), PPARγ agonist (e.g., ciprofibrate and fenobibrate) and PPAR pan-agonists (e.g., GSK 667954) (b) biguanides, e.g., metforminutes, (c) insulin secretagogues, for example, sulphonyl ureas (e.g., glimeperide) and non-sulphonyl ureas (e.g., repaglinide), (d) α-glucosidase inhibitors, e.g., acarbose, (e) protein tyrosine phosphatase-1B inhibitors, (f) glucokinase activators, e.g. PSN105 (g) inhibitors of 11β-hydroxysteroid dehydrogenase type 1, (h) glucagon receptor antagonists, (i) GLP-1 and GLP-1 receptor agonists, e.g. Exenatide (j) insulin or insulin mimetics, (k) GIP and GIP receptor agonists (l) PACAP and PACAP receptor agonists;
2) lipid modulating agents, (i) HMG-CoA reductase inhibitors, e.g., atorvastatin, simvastatin, and fluvastatin. (ii) sequestrants (cholestyramine, colestipol, and dialkylaminoalkyl derivatives of a cross-linked dextran) (iii) nicotinyl alcohol, nicotinic acid or a salt thereof, (iv) inhibitors of cholesterol absorption, e.g., β-sitosterol and ezetimibe, (v) acyl CoA:cholesterol acyltransferase inhibitors, e.g., avasimibe (vi) ileal bile acid transporter inhibitors, and (vii) CETP inhibitors, e.g., torcetrapib;
3) antiobesity compounds, (i) CB1 receptor inverse agonists and antagonists, e.g., rimonabant (ii) β3 adrenergic receptor agonists, (iii) melanocortin-receptor agonists, in particular, melanocortin-4 receptor agonists, (iv) ghrelin antagonists, (v) neuropeptide Y1 or Y5 antagonists, (vi) melanin-concentrating hormone (MCH) receptor antagonists and (vii) fenfluramine, dexfenfluramine, phentermine, sibutramine, and orlistat.
4) antihypertensive agents, (i) ACE inhibitors, e.g., enalapril, lisinopril, and quinapril, (ii) angiotensin II receptor antagonists, e.g., losartan, candesartan, irbesartan, valsartan, and eprosartan, (iii) β-blockers, and (iv) calcium channel blockers; and
5) anti-TNF agent or c-AMP raising agent like PDE inhibitors.

The following terms, used in the specification and claims, shall have the following meanings for the purpose of this application.

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulphinyl, sulphonyl group or —NR_{α}—, wherein R_{α}, can be hydrogen, alkyl, cycloalkyl, alkenyl, cycloalkenyl, alkynyl, aryl, acyl, aralkyl, —C(═O)OR_{λ}, SOₘR_{ψ} or —C(═O)NR_{λ})R_{π}. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl, heterocyclyl, heteroaryl, (heterocyclyl)alkyl, cycloalkoxy, —CH═N—O(C₁₋₆alkyl), —CH═N—NH(C₁₋₆alkyl), —CH═N—NH(C₁₋₆alkyl)-C₁₋₆alkyl, arylthio, thiol, alkylthio, aryloxy, nitro, aminosulfonyl, aminocarbonylamino, —NHC(═O)R_{λ}), —NR_{λ}R_{π}, —C(═O)NR_{λ}R_{π}, —NHC(═O)NR_{λ})R_{π}, —C(═O)heteroaryl, C(═O)heterocyclyl, —O—C(═O)NR_{λ})R_{π} {wherein R_{λ} and R_{π} are independently selected from hydrogen, halogen, hydroxy, alkyl, alkenyl, alkynyl, alkenyl, alkoxy, cycloalkyl, cycloalkenyl, aryl, aralkyl, heterocyclyl, heteroaryl, heterocyclylalkyl, heteroarylalkyl or carboxy}, nitro or —SOₘR_{ψ} {wherein m is an integer from 0-2 and R_{ψ} is hydrogen, alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —NR_{λ}R_{π}, —C(═O)NR_{λ})R_{π}, —OC(═O)NR_{λ}R_{π}, —NHC(═O)NR_{λ}R_{π}, hydroxy, alkoxy, halogen, CF₃, cyano, and —SOₘR_{ψ}; or an alkyl group also may be interrupted by 1-5 atoms of groups independently selected from oxygen, sulfur or —NR_{α}— (wherein R_{α}, R_{λ}, R_{π}, m and R_{ψ}, are the same as defined earlier). Unless otherwise constrained by the definition, all substituents may be substituted further by 1-3 substituents selected from alkyl, alkenyl, alkynyl, carboxy, carboxyalkyl, —NR_{λ}R_{π}, —C(═O)NR_{λ}R_{π}, —O—C(═O)NR_{λ}R_{π}, hydroxy, alkoxy, halogen, CF₃, cyano, and —SOₘR_{ψ} (wherein R_{λ}, R_{π}, m and R_{ψ}, are the same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans or geminal geometry. Alkenyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkenyl is attached to a heteroatom, the double bond cannot be alpha to the heteroatom. Alkenyl groups may be substituted further with one or more substituents selected from alkyl, alkenyl, alkynyl, alkoxy, cycloalkyl, cycloalkenyl, acyl, acylamino, acyloxy, —NHC(═O)R_{λ}), —NR_{λ}R_{π}, —C(═O)NR_{λ}R_{π}, —NHC(═O)NR_{λ}R_{π}, —O—C(═O)NR_{λ}R_{π}, alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro or SOₘR_{ψ (wherein R}_{λ}, R_{π}, m and R_{ψ} are as defined earlier). Unless otherwise constrained by the definition, alkenyl substituents optionally may be substituted further by 1-3 substituents selected from alkyl, alkenyl, alkynyl, carboxy, hydroxy, alkoxy, halogen, —CF₃, cyano, —NR_{λ}R_{π}, —C(═O)NR_{λ}R_{π}, —O—C(═O)NR_{λ}R_{π} and —SOₘR_{ψ} (wherein R_{λ}, R_{π}, m and R_{ψ} are as defined earlier). Groups, such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkynyl,” unless otherwise specified, refers to a monoradical of an unsaturated hydrocarbon, having from 2 to 20 carbon atoms. Alkynyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkynyl groups are attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)R_{λ}, —NR_{λ}R_{π}, —NHC(═O)NR_{λ}R_{π}, —C(═O)NR_{λ}R_{π}, —O—C(═O)NR_{λ}R_{π}, or —SOₘR_{ψ} (wherein R_{λ}, R_{π}, m and R_{ψ} are the same as defined earlier). Unless otherwise constrained by the definition, alkynyl substituents optionally may be substituted further by 1-3 substituents selected from alkyl, alkenyl, alkynyl, carboxy, carboxyalkyl, hydroxy, alkoxy, halogen, CF₃, —NR_{λ}R_{π}, —C(═O)NR_{λ}R_{π}, —NHC(═O)NR_{λ}R_{π}, —C(═O)NR_{λ}R_{π}, cyano or —SOₘR_{ψ} (wherein R_{λ}, R_{π}, m and R_{ψ} are the same as defined earlier).

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like or multiple ring structures, including adamantanyl, and bicyclo [2.2.1]heptane or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{λ}R_{π}, —NHC(═O)NR_{λ}R_{π}, —NHC(═O)R_{λ}, —C(═O)NR_{λ}R_{π}, —O—C(═O)NR_{λ}R_{π}, nitro, heterocyclyl, heteroaryl, heterocyclylalkyl, heteroarylalkyl or SOₘR_{ψ} (wherein R_{λ}R_{π}, m and R_{ψ} are the same as defined earlier). Unless otherwise constrained by the definition, cycloalkyl substituents optionally may be substituted further by 1-3 substituents selected from alkyl, alkenyl, alkynyl, carboxy, hydroxy, alkoxy, halogen, CF₃, —NR_{λ}R_{π}, —C(═O)NR_{λ}R_{π}, —NHC(═O)NR_{λ}R_{π}, —OC(═O)NR_{λ}R_{π}, cyano or —SOₘR_{ψ} (wherein R_{λ}, R_{π}, m and R_{ψ} are the same as defined earlier). “Cycloalkylalkyl” refers to alkyl-cycloalkyl group linked through alkyl portion, wherein the alkyl and cycloalkyl are the same as defined earlier.

The term “aryl,” unless otherwise specified, refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COOR_{ψ}, NHC(═O)R_{λ}), —NR_{λ}R_{π}, —C(═O)NR_{λ}R_{π}, —NHC(═O)NR_{λ}R_{π}, —O—C(═O)NR_{λ}R_{π}, —SOₘR_{ψ}, carboxy, heterocyclyl, heteroaryl, heterocyclylalkyl, heteroarylalkyl or amino carbonyl amino, mercapto, haloalkyl, optionally substituted aryl, optionally substituted heterocyclylalkyl, thioalkyl, —CONHR_{π}, —OCOR_{π}, —COR_{π}, —NHSO₂R_{π} or —SO₂NHR_{π} (wherein R_{λ}), R_{π}, m and R_{ψ} are the same as defined earlier). Aryl groups optionally may be fused with a cycloalkyl group, wherein the cycloalkyl group may optionally contain heteroatoms selected from O, N or S. Groups such as phenyl, naphthyl, anthryl, biphenyl, and the like exemplify this term.

The term “heteroaryl,” unless otherwise specified, refers to an aromatic ring structure containing 5 or 6 ring atoms or a bicyclic or tricyclic aromatic group having from 8 to 10 ring atoms, with one or more heteroatom(s) independently selected from N, O or S optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{λ}R_{π}, CH═NOH, —(CH₂)_{w}C(═O)R_{η} {wherein w is an integer from 0-4 and R_{η} is hydrogen, hydroxy, OR_{λ}, NR_{λ}R_{π}, —NHOR_{ω} or —NHOH}, —C(═O)NR_{λ}R_{π}—NHC(═O)NR_{λ}R_{π}, —SOₘR_{ψ}, —O—C(═O)NR_{λ}R_{π}, —O—C(═O)R_{λ}, or —O—C(═O)OR_{λ} (wherein m, R_{ψ}, R_{λ} and R_{π} are as defined earlier and R_{ω} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzthiazinyl, benzthiazinonyl, benzoxazinyl, benzoxazinonyl, quinazonyl, carbazolyl phenothiazinyl, phenoxazinyl, benzothiazolyl or benzoxazolyl, and the like.

The term “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, optionally substituted aryl, alkoxy, alkaryl, cyano, nitro, oxo, carboxy, optionally substituted heterocyclyl, optionally substituted heterocyclylalkyl, optionally substituted heteroaryl, —O—C(═O)R_{λ}, —O—C(═O)OR_{λ}), —C(═O)NR_{λ}R_{π}, SOₘR_{ψ}, —O—C(═O)NR_{λ}R_{π}, —NHC(═O)NR_{λ}R_{π}, —NR_{λ}R_{π}, mercapto, haloalkyl, thioalkyl, —COOR_{ψ}, —COONHR_{λ}, —COR_{λ}, —NHSO₂R_{λ} or SO₂NHR_{λ} (wherein m, R_{ψ}, R_{λ} and R_{π} are as defined earlier) or guanidine. Heterocyclyl can optionally include rings having one or more double bonds. Such ring systems can be mono-, bi- or tricyclic. Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, benzoxazinyl, benzthiazinyl, imidazolyl, benzimidazolyl, tetrazolyl, carbaxolyl, indolyl, phenoxazinyl, phenothiazinyl, dihydropyridinyl, dihydroisoxazolyl, dihydrobenzofuryl, azabicyclohexyl, thiazolidinyl, dihydroindolyl, pyridinyl, isoindole 1,3-dione, piperidinyl, tetrahydropyranyl, piperazinyl, 3H-imidazo[4,5-b]pyridine, isoquinolinyl, 1H-pyrrolo[2,3-b]pyridine or piperazinyl and the like.

The term “carboxy,” as defined herein, refers to —C(═O)OH.

The term “amino” refers to —N(Rᵢ)₂, (wherein each Rᵢ is independently selected from hydrogen, alkyl, cycloalkyl, aryl, heteroaryl, heterocyclyl).

“Acyl” refers to —C(═O)R″ wherein R″ is selected from hydrogen, alkyl, cycloalkyl, aryl, aralkyl, heteroaryl, heterocyclyl, heteroarylalkyl or heterocyclylalkyl.

The term “halo” refers to —F, —Cl, —Br, and —I.

The term “leaving group” refers to groups that exhibit or potentially exhibit the properties of being labile under the synthetic conditions and also, of being readily separated from synthetic products under defined conditions. Examples of leaving groups include, but are not limited to, halogen (e.g., F, Cl, Br, I), triflates, tosylate, mesylates, alkoxy, thioalkoxy, or hydroxy radicals and the like.

The term “protecting groups” refers to moieties that prevent chemical reaction at a location of a molecule intended to be left unaffected during chemical modification of such molecule. Unless otherwise specified, protecting groups may be used on groups, such as hydroxy, amino, or carboxy. Examples of protecting groups are found in T. W. Greene and P. G. M. Wuts, “Protective Groups in Organic Synthesis”, 2^{nd} Ed., John Wiley and Sons, New York, N.Y., which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule.

The term “pharmaceutically acceptable salts” refers to derivatives of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like. The term “pharmaceutically acceptable salts” refer to a salt prepared from pharmaceutically acceptable non-toxic inorganic or organic acid. Examples of such inorganic acids include, but are not limited to, hydrochloric, hydrobromic, hydroiodic, nitrous, nitric, carbonic, sulfuric, phosphoric acid, and the like. Appropriate organic acids include, but are not limited to aliphatic, cycloaliphatic, aromatic, heterocyclic, carboxylic and sulfonic classes of organic acids, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic, methanesulfonic, ethanesulfonic, benzenesulfonic, panthenic, toluenesulfonic, 2-hydroxyethanesulfonic acid and the like.

The term “pharmaceutically acceptable solvates” refers to solvates with water (i.e., hydrates) or pharmaceutically acceptable solvents, for example solvates with ethanol and the like. Such solvates are also encompassed within the scope of the disclosure. Furthermore, some of the crystalline forms for compounds described herein may exist as polymorphs and as such are intended to be included in the scope of the disclosure.

The present invention within its scope also includes prodrugs of these agents. In general, such prodrugs will be functional derivatives of these compounds, which are readily convertible in vivo into the active drugs. Conventional procedure for the selection and preparation of suitable prodrug derivatives are described, for example, in “Targeted prodrug design to optimize drug delivery”, AAPS PharmSci. (2000), 2(1), E6.

### DETAILED DESCRIPTION OF THE INVENTION

The compounds disclosed herein may be prepared by techniques well known in the art and familiar to the skilled synthetic organic chemist. In addition, the compounds of the present invention may be prepared by the following reaction sequences as depicted in, for example, Schemes I to V.

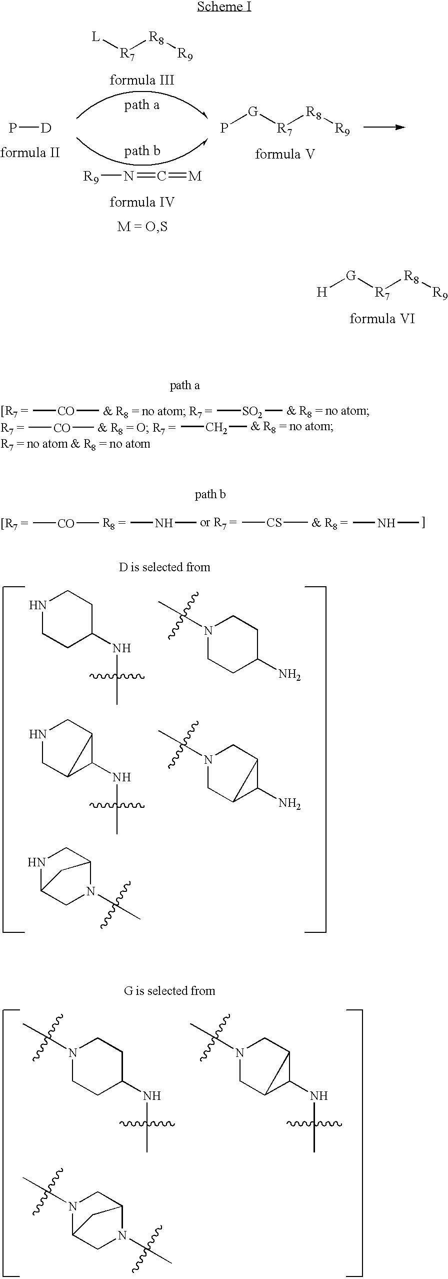

The compounds of formula VI can be prepared, for example, by following ‘Scheme I’.

Path a: The compound of formula II [wherein P is an amino protecting group selected from Boc, Fmoc, allyloxycarbonyl, benzyl, and Cbz] can be reacted with a compound of formula III (wherein L is a leaving group such as halide; R₇, R₈ and R₉ are defined as earlier) to give the compound of formula V. The compound of formula V on deprotection can yield a compound of formula VI.
Path b: The compound of formula II can be reacted with a compound of formula IV (wherein M is O or S, and R₉ is defined as earlier) to form a compound of formula V. The compound of formula V on deprotection can yield a compound of formula VI.

The reaction of the compound of formula II with the compound of formula III (wherein R₇ is —CH₂—, —CO— or —SO₂— and R₈ is —O— or no atom) to give the compound of formula V (Path a) can be carried out in a solvent, for example, dichloromethane, toluene, dichloroethane, tetrahydrofuran, ether or dioxane and in the presence of a base, for example, triethylamine, diisopropylethylamine or N-methylmorpholine at a temperature of 0 to 100° C.

The reaction of the compound of formula II with a compound of formula III (wherein R₇ and R₈ are no atom) to give a compound of formula V (Path a) can be carried out in a solvent, for example, dimethylformamide, dioxane, tetrahydrofuran or dimethylsulphoxide and in the presence of a base, for example, potassium carbonate, triethylamine or N,N-diisopropylethylamine at a temperature of 0 to 150° C.

The reaction of the compound of formula II with the compound of formula IV to give a compound of formula V (Path b) can be carried out in a solvent, for example, dichloromethane, toluene, dichloroethane, tetrahydrofuran, ether or dioxane and, optionally, in the presence of a base, for example, potassium carbonate, triethylamine, diisopropylethylamine or N-methylmorpholine.

The deprotection of the compound of formula V to form the compound of formula VI can be carried out under acidic (e.g., p-toluenesulphonic acid or trifluoroacetic acid) or basic (e.g., piperidine) conditions in a solvent for example, acetonitrile, tetrahydrofuran or dioxane, dimethylformamide or a mixture thereof. The deprotection can also be carried out by other deprotection methods known to a skilled organic chemist.

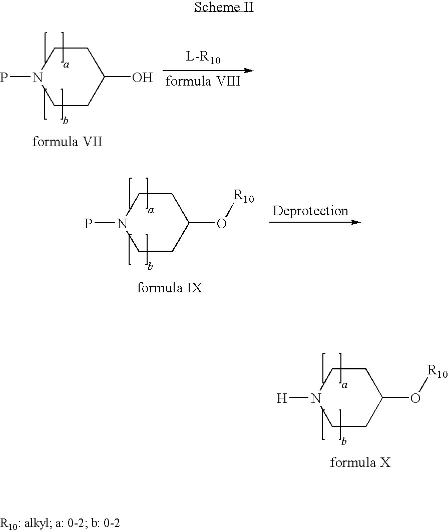

The compounds of formula X can be prepared, for example, by following ‘Scheme II’.

The compound of formula VII (wherein P is previously defined) can be reacted with a compound of formula VIII (wherein L is a leaving group such as halide and R₁₀ is alkyl) to give a compound of formula IX, which on deprotection can give a compound of formula X.

The reaction of the compound of formula VII with the compound of formula VIII to give the compound of formula IX can be carried out in a solvent, for example, tetrahydrofuran, dimethyl formamide, dimethylsulphoxide or dichloromethane and in the presence of a base, for example, sodium hydride, n-butyl lithium or silver carbonate at a temperature of −78 to 50° C. The deprotection of compound of formula IX can be carried out as that of the deprotection of the compound of formula V.

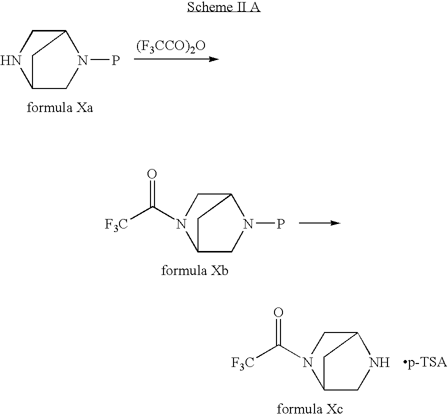

The compound of formula Xc can be prepared, for example, by following Scheme II A.

The compound of formula Xa (wherein P is previously defined) can be reacted with trifluoroacetic anhydride to form a compound of formula Xb, which can then be deprotected to form a compound of formula Xc.

The reaction of compound of formula Xa with trifluoroacetic anhydride to form a compound of formula Xb can be carried out in the presence of one or more bases, for example, triethylamine, potassium carbonate or N,N-diisopropylethylamine in one or more halogenated solvents such as dichloromethane, dichloroethane, chloroform, carbon tetrachloride, etc

The deprotection of compound of formula Xb to form a compound of formula Xc can be carried out as that of deprotection of compound of Formula V.

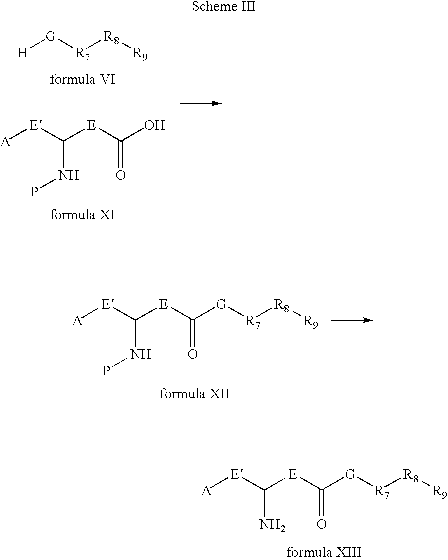

The compound of formula XIII can be prepared, for example, by following ‘Scheme III’. Thus the compound of formula VI is reacted with a compound of formula XI (wherein P is an amino protecting group and A, E, and E′ are defined as earlier) to form a compound of formula XII, which is deprotected to give a compound of formula XIII.

The reaction of the compound of formula VI with a compound of formula XI to give a compound of formula XII can be carried out in a solvent, for example, tetrahydrofuran, dimethylformamide or dioxane using a coupling agent, for example, 1,3-dicyclo-hexylcarbodiimide (DCC), 1-ethyl-3-(3′-dimethylaminopropyl)carbodiimide hydrochloride (EDCI), N-[(dimethylamino)-1H-1,2,3-triazolo[4,5-b]pyridylmethylene]-N-methylmethanaminium hexafluorophosphate N-oxide (HATU) or benzotriazol-1-yl-N-oxy-tris(pyrrolidino)phosphonium hexafluorophosphate (PyBOP) and, optionally, a catalyst, for example, 1-hydroxybenzotriazole (HOBt), 3-hydroxy-3,4-dihydro-4-oxo-1,2,3-benzotriazine (HODhbt) or 7-aza-1-hydroxybenzo-triazole (HOAt) and, optionally, with a base, for example, N,N-dimethylaminopyridine (DMAP), triethylamine, N,N-diisopropylethylamine or N-methylmorpholine. The reaction can also be carried out by any other method well known for amide bond formation. The deprotection of the compound of formula XII to form the compound of formula XIII can be carried out as that of the deprotection of the compound of formula V.

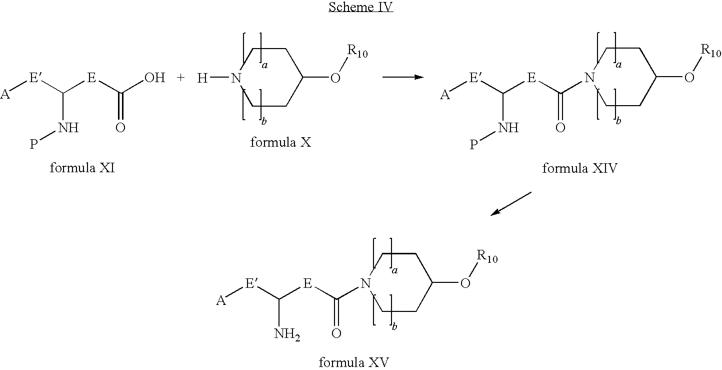

The compound of formula XV can be prepared, for example, by following ‘Scheme IV’.

Thus the compound of formula XI can be reacted with a compound of formula X (using the conditions similar to the coupling of the compounds of formulas VI and XI) to form a compound of formula XIV. The later compound can be deprotected to give a compound of formula XV (using the conditions similar to that of the deprotection of the compound of formula V).

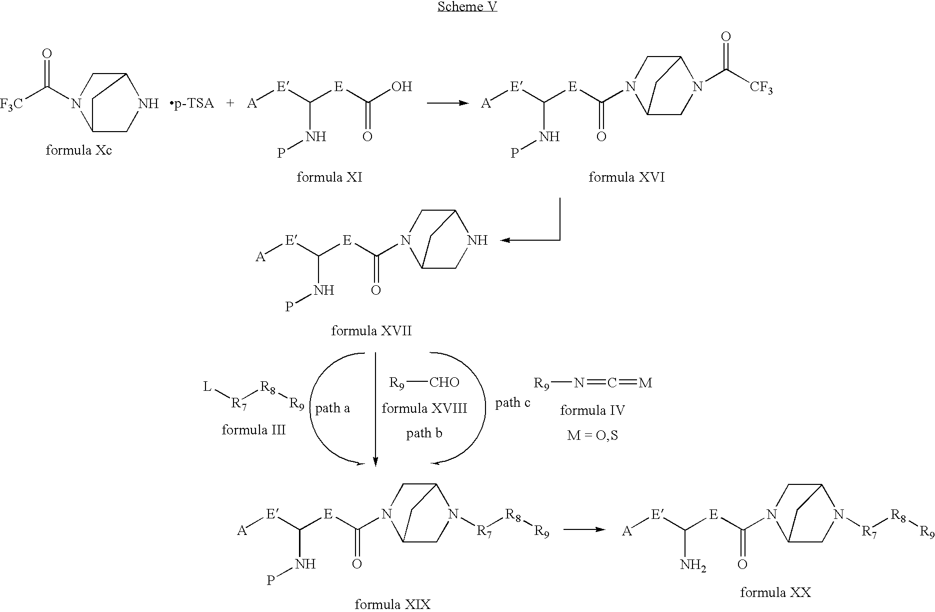

The compound of formula XX can be prepared, for example, by following Scheme V.

Thus, compound of formula X c can be reacted with compound of formula XI to form a compound of formula XVI, which can then be deprotected to form a compound of formula XVII. The compound of formula XVII can be reacted through three pathways to give a compound of formula XIX:

Path a: The compound of formula XVII can be reacted with a compound of formula III (wherein L is a leaving group such as halide; R₇, R₈ and R₉ are defined as earlier) to give the compound of formula XIX;
Path b: The compound of formula XVII can be reacted with a compound of formula XVIII (wherein R₉ is defined as earlier) to give a compound of formula XIX; or
Path c: The compound of formula XVII can be reacted with a compound of formula IV (wherein M is O or S and R₉ is defined as earlier) to form a compound of formula XIX.

The compound of formula XIX can be deprotected to yield a compound of formula XX.

The reaction of compound of formula XI with a compound of formula Xc to form a compound of formula XVI can be carried out in one or more dry solvents, for example, dimethylformamide, tetrahydrofuran or dioxane using a coupling agent, for example, 1-ethyl-3-(3′-dimethylaminopropyl) carbodiimide hydrochloride (EDCI), 1,3-dicyclo-hexylcarbodiimide (DCC), N-[(dimethylamino)-1H-1,2,3-triazolo[4,5-b]pyridylmethylene]-N-methyl methanaminium hexafluorophosphate N-oxide (HATU) or benzotriazol-1-yl-N-oxy-tris(pyrrolidino)phosphonium hexafluorophosphate (PyBOP) in the presence of a peptide coupling agent, for example, 1-hydroxybenzotriazole (HOBt), 3-hydroxy-3,4-dihydro-4-oxo-1,2,3-benzotriazine (HODhbt) or 7-aza-1-hydroxybenzotriazole and, optionally, with a base, for example, triethylamine, N,N-dimethylaminopyridine (DMAP), N,N-diisopropylethylamine or N-methylmorpholine. The reaction can also be carried out by any other amide bond-formation method.

The conversion of the compound of formula XVI to a compound of formula XVII can be carried out under basic (e.g., potassium carbonate, piperidine) or acidic (e.g., p-toluenesulphonic acid and trifluoroacetic acid) conditions in a solvent, for example, methanol, acetonitrile, tetrahydrofuran, dioxane, dimethylformamide, or mixtures thereof.

The reaction of the compound of formula XVII with a compound of formula III (wherein L is a leaving group) to give a compound of formula XIXI (Path a) can be carried out in a solvent, for example, dichloromethane, dimethylformamide, dioxane, tetrahydrofuran or dimethylsulphoxide and in the presence of a base, for example, triethylamine, potassium carbonate, or N,N-diisopropyl-ethylamine.

The reductive amination of the compound of formula XVII with a compound of formula XVIII to give a compound of formula XIX (Path b) can be carried out in the presence of one or more reducing agents, for example, sodium triacetoxyborohydride, sodium cyanoborohydride or sodium borohydride in one or more chlorinated solvent, for example, dichloromethane, chloroform or carbon tetrachloride, polar protic solvents, for example, methanol, ethanol, propanol, isopropanol, water or polar aprotic solvent, for example, acetonitrile, or mixtures thereof.

The reaction of the compound of formula XVII with the compound of formula IV to give a compound of formula XIX (Path c) can be carried out in a solvent, for example, dichloromethane, toluene, dichloroethane, tetrahydrofuran, ether or dioxane, and optionally, in the presence of a base, for example, triethylamine, potassium carbonate, diisopropylethylamine or N-methylmorpholine.

The deprotection of the compound of formula XIX to form the compound of formula XX can be carried out under similar conditions as that of deprotection of compound of formula V.

In the above schemes, where specific bases, acids, solvents, coupling agents, protecting groups, hydrolyzing agents, etc., are mentioned, it is to be understood that other acids, bases, solvents, coupling agents, protecting groups, hydrolyzing agents, etc., known to those skilled in the art may also be used. Similarly, the reaction temperature and duration of the reactions may be adjusted according to the requirements that arise during the process.

The examples set forth below demonstrate the general synthetic procedures for the preparation of representative compounds. The examples are provided to illustrate some particular aspects of the disclosure and do not limit the scope of the present invention.

### EXPERIMENTAL

### Synthesis of 4-{N-(2,4-difluorobenzenesulphonyl)}amino-1-piperidine (pTSA Salt)

### a. Step a: Synthesis of 4-{N-(2,4-difluorobenzenesulphonyl)}amino-1-(tert-butyloxycarbonyl)-piperidine

To a solution of 4-amino-1-(tert-butyloxycarbonyl)piperidine (1.000 g, 5.00 mmol) and triethylamine (0.15 mL, 10.5 mmol) in dichloromethane (10.0 mL) at 0° C., was added dropwise a solution of 2,4-difluorobenzenesulphonyl chloride (0.87 mL, 6.50 mmol) in dichloromethane (5.0 mL). The reaction mixture was stirred at room temperature for about 2-3 hours and partitioned between water (10.0 mL) and dichloromethane (15.0 mL). The aqueous layer was extracted with dichloromethane (15.0 mL). The combined organic layer was washed water and brine, dried over anhydrous sodium sulphate and concentrated under reduced pressure to yield the title compound (1.650 g), which was used as such in the next step.

¹H NMR (300 MHz, CDCl₃): 1.20-1.50 (m, 1H), 1.70-1.85 (m, 2H), 2.7-2.9 (m, 2H), 3.25-3.45 (m, 1H), 3.8-4.05 (m, 2H), 4.69 (d, 1H, J=7.8 Hz), 6.9-7.1 (m, 2H), 7.80-8.00 (m, 1H);

ESI-MS (m/z): 377.1 (M⁺+1).

### b. Step b: 4-{N-(2,4-difluorobenzenesulphonyl)}amino-1-piperidine (p TSA Salt)

To the compound (1.500 g, 4.18 mmol) obtained from ‘step a’ in acetonitrile (15.0 mL), was added p-toluenesulphonic acid (1.23 g, 6.49 mmol). The mixture was stirred for 12 hours at room temperature. The solvent was evaporated and the residue taken in ethyl acetate. The mixture was stirred for 30 minutes, and the precipitated solid filtered, washed with cold ethyl acetate and dried to yield the title compound (1.760 g, 82%).

¹H NMR (400 MHz, MeOH-d4): δ 1.62-1.80 (m, 2H), 1.90-2.05 (m, 2H), 2.36 (s, 3H), 2.95-3.10 (m, 2H), 3.25-3.35 (m, 2H), 3.40-3.55 (s, 1H), 7.05-7.30 (m, 4H), 7.69 (d, 2H, J=7.8 Hz), 7.85-8.00 (m, 1H); ESI-MS (m/z): 277 (M⁺+1, free amine).

The following intermediates were prepared by following the preparation of 4-{N-(2,4-difluorobenzenesulphonyl)}amino-1-piperidine (pTSA salt) with the use of appropriate amine [4-(N-tert-butyloxycarbonyl)amino]piperidine, 4-amino-1-(tert-butyloxycarbonyl)piperidine, 6-(tert-butyloxycarbonyl)amino-3-azabicyclo [3.1.0]hexane or 2-(tert-butyloxycarbonyl)-2,5-diazabicyclo[2.2.1]heptane] and electrophile [acyl chloride, sulphonyl chloride or chloroformate]. In those cases, where the solid didn't precipitate (semi-solid) in step b, the solvent was decanted. Fresh ethyl acetate was added and, after stirring for 5 minutes, the solvent was decanted and the resulting semi-solid was dried under vacuum to afford the pure product.
4-Amino-1-(4-fluorobenzoyl)piperidine (pTSA salt)

[ESI-MS (m/z): 223.2 (M⁺+1), free amine];
4-Amino-1-(4-fluorobenzenesulphonyl)piperidine (pTSA salt)

[ESI-MS (m/z): 259.1 (M⁺+1), free amine];
4-Amino-1-{morpholin-1-carbonyl}piperidine (pTSA salt)

[ESI-MS (m/z): 214.3 (M⁺+1), free amine];
4-Amino-1-(methanesulphonyl)piperidine (pTSA salt)

[ESI-MS (m/z): 179 (M⁺+1), free amine];
4-(N-[4-Fluorobenzoyl])amino-1-piperidine (pTSA salt)

[ESI-MS (m/z): 223.2 (M⁺+1), free amine];
4-(N-[4-Fluorobenzenesulphonyl])amino-1-piperidine (pTSA salt)

[ESI-MS (m/z): 259.0 (M⁺+1), free amine];
4-(N-[Morpholin-1-carbonyl])amino-1-piperidine (pTSA salt)

[ESI-MS (m/z): 214 (M⁺+1), free amine];
4-(N-[Thiophene-2-carbonyl])amino-1-piperidine (pTSA salt)

[ESI-MS (m/z): 211 (M⁺+1), free amine];
4-(N-[Cyclopentyl-1-carbonyl])amino-1-piperidine (pTSA salt)

[ESI-MS (m/z): 197 (M⁺+1), free amine];
4-(N-[4-Cyanobenzenesulphonyl])amino-1-piperidine (pTSA salt)

[ESI-MS (m/z): 266 (M⁺+1), free amine];
4-(N-[Propan-2-sulphonyl])amino-1-piperidine (pTSA salt)

[ESI-MS (m/z): 208.22 (M⁺+1), free amine];
4-(N-[3-Fluoro-4-methoxybenzoyl])amino-1-piperidine (pTSA salt)

[ESI-MS (m/z): 253.15 (M⁺+1), free amine];
3-(Thiophen-2-sulphonyl)azabicyclo[3.1.0]hexan-6-amine (pTSA salt)

[ESI-MS (m/z): 245.1 (M⁺+1), free amine];
3-(4-Cyanobenzoyl)azabicyclo[3.1.0]hexan-6-amine (pTSA salt)

[ESI-MS (m/z): 360.41 (M⁺+1), free amine];
3-(2,6-Difluorobenzoyl)azabicyclo[3.1.0]hexan-6-amine (pTSA salt)

[ESI-MS (m/z): 370.51 (M⁺+1), free amine];
3-(4-Trifluorobenzenesulphonyl)azabicyclo[3.1.0]hexan-6-amine (pTSA salt)

[ESI-MS (m/z): 307.1 (M⁺+1), free amine];
3-(Thiophene-2-carbonyl)azabicyclo[3.1.0]hexan-6-amine (pTSA salt)

[ESI-MS (m/z): 209.1 (M⁺+1), free amine];
3-(Ethanesulphonyl)azabicyclo[3.1.0]hexan-6-amine (pTSA salt)

[ESI-MS (m/z): 191.2 (M⁺+1), free amine];
3-(4-Methylbenzenesulphonyl)azabicyclo[3.1.0]hexan-6-amine (pTSA salt)

[ESI-MS (m/z): 253.2 (M⁺+1), free amine];
4-(N-[2,2,2-Trifluoroethanesulphonyl])amino-1-piperidine (pTSA salt)
4-(N-[4-Cyanobenzenesulfonyl])amino-1-piperidine (pTSA salt)

[ESI-MS (m/z): 265.96 (M⁺+1), free amine];
4-(N-[2,4-Difluorobenzenesulfonyl])amino-1-piperidine (pTSA salt)

[ESI-MS (m/z): 277.01 (M⁺+1), free amine];
4-(N-[4-Methyl-2-methoxycarbonylthiophen-5-ylsulfonyl])amino-1-piperidine (pTSA salt)

[ESI-MS (m/z): 319 (M⁺+1), free amine];
4-(N-[1,3,5-Trimethylpyrazol-4-ylsulfonyl])amino-1-piperidine (pTSA salt)

[ESI-MS (m/z): 273.19 (M⁺+1), free amine];
4-(N-[2,5-Dimethyl-3-methoxycarbonylfuran-4-ylsulfonyl])amino-1-piperidine (pTSA salt)

[ESI-MS (m/z): 317.18 (M⁺+1), free amine];
4-(N-[4-Fluorobenzenesulfonyl])amino-1-piperidine (pTSA salt)

[ESI-MS (m/z): 259.10 (M⁺+1), free amine];
4-(N-[4-Acetylbenzenesulfonyl])amino-1-piperidine (pTSA salt)

[ESI-MS (m/z): 283.06 (M⁺+1), free amine];
4-(N-[2,4-Difluorobenzenesulfonyl])amino-1-piperidine (pTSA salt)

[ESI-MS (m/z): 277.10 (M⁺+1), free amine];
3-(N-Methylsulfonyl)amino-1-piperidine (pTSA salt)

[ESI-MS (m/z): 179.38 (M⁺+1), free amine];
3-(N-[4-Methyl-2-methoxycarbonyl thiophen-5-ylsulfonyl])amino-1-piperidine (pTSA salt)

[ESI-MS (m/z): 319 (M⁺+1), free amine];
3-(N-Isopropylsulfonyl)amino-1-piperidine (pTSA salt)

[ESI-MS (m/z): 207 (M⁺+1), free amine];
3-(N-Ethylsulfonyl)amino-1-piperidine (pTSA salt)

[ESI-MS (m/z): 193 (M⁺+1), free amine];
4-(N-Propanoyl)amino-1-piperidine (pTSA salt)

[ESI-MS (m/z): 157.23 (M⁺+1), free amine];
4-(N-Ethanoyl)amino-1-piperidine (pTSA salt)

[ESI-MS (m/z): 143.01 (M⁺+1), free amine];
4-(N-[3-Fluorobenzoyl])amino-1-piperidine (pTSA salt)

[ESI-MS (m/z): 223.12 (M⁺+1), free amine];
4-(N-[3,5-Difluoro-2-cyanophenyl])amino-1-piperidine (pTSA salt)

[ESI-MS (m/z): 238.09 (M⁺+1), free amine];
4-(N-Cyanophenyl)amino-1-piperidine (pTSA salt)

[ESI-MS (m/z): 202.12 (M⁺+1), free amine];
4-(N-[3,5-Difluoro-4-cyanophenyl])amino-1-piperidine (pTSA salt)

[ESI-MS (m/z): 238.09 (M⁺+1), free amine];
3-(N-[3,5-Difluoro-4-cyanophenyl])amino-1-piperidine (pTSA salt)

[ESI-MS (m/z): 238.16 (M⁺+1), free amine];
3-(N-[1-Cyanophenyl])amino-1-piperidine (pTSA salt)

[ESI-MS (m/z): 202.19 (M⁺+1), free amine];
3-(N-[4-Cyanophenyl])amino-1-piperidine (pTSA salt)

[ESI-MS (m/z): 202.19 (M⁺+1), free amine];
4-(N-[4-Cyanophenyl])amino-1-piperidine (pTSA salt)

[ESI-MS (m/z): 201.19 (M⁺+1), free amine];
4-(N-[2-Trifluoromethyl-4-cyanophenyl])amino-1-piperidine (pTSA salt)

[ESI-MS (m/z): 270.11 (M⁺+1), free amine];
2-Acetyl-2,5-diazabicyclo [2.2.1]heptane (pTSA salt)

[ESI-MS (m/z): 141 (M⁺+1), free amine];
2-[(4-Fluorophenyl)sulfonyl]-2,5-diazabicyclo[2.2.1]heptane (pTSA salt)

[ESI-MS (m/z): 257.11 (M⁺+1), free amine];
2-(Ethylsulfonyl)-2,5-diazabicyclo [2.2.1]heptane (pTSA salt)

[ESI-MS (m/z): 191 (M⁺+1), free amine];
2-(4-Cyano-3-trifluoromethylphenyl)-2,5-diazabicyclo[2.2.1]heptane (pTSA salt)

[ESI-MS (m/z): 268 (M⁺+1), free amine];
2-(4-Fluorobenzoyl)-2,5-diazabicyclo[2.2.1]heptane (pTSA salt)

[ESI-MS (m/z): 221 (M⁺+1), free amine];
2-(4-Fluorophenylaminocarbonyl)-2,5-diazabicyclo[2.2.1]heptane (pTSA salt)

[ESI-MS (m/z): 221.18 (M⁺+1), free amine];
2-(2-Thienylsulfonyl)-2,5-diazabicyclo[2.2.1]heptane (pTSA salt)

[ESI-MS (m/z): 245.20 (M⁺+1), free amine];
2-(4-Cyanophenylsulfonyl)-2,5-diazabicyclo[2.2.1]heptane (pTSA salt)

[ESI-MS (m/z): 264.24 (M⁺+1), free amine];
2-[(3,5-Difluorophenyl)sulfonyl]-2,5-diazabicyclo [2.2.1]heptane (pTSA salt)

[ESI-MS (m/z): 275.22 (M⁺+1), free amine];
2-(Propylsulfonyl)-2,5-diazabicyclo [2.2.1]heptane (pTSA salt)

[ESI-MS (m/z): 205.25 (M⁺+1), free amine];
2-(Isopropylsulfonyl)-2,5-diazabicyclo[2.2.1]heptane (pTSA salt)

[ESI-MS (m/z): 205.24 (M⁺+1), free amine];
2-(Butylsulfonyl)-2,5-diazabicyclo[2.2.1]heptane (pTSA salt)

[ESI-MS (m/z): 219 (M⁺+1), free amine];
2-(Phenylsulfonyl)-2,5-diazabicyclo[2.2.1]heptane (pTSA salt)

[ESI-MS (m/z): 239 (M⁺+1), free amine];
2-{[4-(Trifluoromethoxy)phenyl]sulfonyl}-2,5-diazabicyclo[2.2.1]heptane (pTSA salt)

[ESI-MS (m/z): 323 (M⁺+1), free amine]; and
2-(Methylsulfonyl)-2,5-diazabicyclo[2.2.1]heptane (pTSA salt)

[ESI-MS (m/z): 177.02 (M⁺+1), free amine]. Synthesis of 4-amino-1-[{N-(4-chlorophenyl)}aminocarbonyl]piperidine (pTSA salt)

### a. Step a: Synthesis of 4-[(N-tert-butyloxycarbonyl)amino]-1-[{N-(4-chlorophenyl)}amino carbonyl]piperidine

To a solution of 4-[(N-tert-butyloxycarbonyl)amino]piperidine (0.500 g, 2.50 mmol) in dichloromethane (10.0 mL) at 0° C., was added dropwise a solution of 4-chlorophenyl isocyanate (0.38 mL, 3.0 mmol) in dichloromethane (5.0 mL). The mixture was stirred at RT for about 3 hours and partitioned between water (10.0 mL) and dichloromethane (20.0 mL). The organic layer was washed with brine, dried over anhydrous sodium sulphate and concentrated under reduced pressure to yield the title product, which was used directly in the next step.

¹H NMR (400 MHz, MeOH-d4): δ 1.25-1.50 (m, 11H), 1.60 (s, 1H), 2.01 (d, 2H, J=8.0 Hz), 2.92-3.05 (m, 2H), 3.64-3.82 (br s, 1H), 3.99 (d, 2H, J=12.0 Hz), 4.40-4.55 (br s, 1H), 7.15-7.3 (m, 4H); ESI-MS (m/z): 376 (M⁺+23).

### b. Step b: Synthesis of 1-[{N-(4-chlorophenyl)}aminocarbonyl]-4-aminopiperidine (pTSA Salt)

To the compound obtained from ‘step a’ in acetonitrile (7.0 mL), was added p-toluenesulphonic acid (0.713 g, 3.75 mmol) at room temperature. The reaction mixture was stirred for 12 hours. The solvent was evaporated and the crude mixture taken in ethyl acetate and stirred for 30 minutes. The precipitate was filtered, washed with cold ethyl acetate and dried under reduced pressure to yield the title compound (0.744 g, 70%)

¹H NMR (400 MHz, MeOH-d4): δ 1.53-1.60 (m, 2H), 2.02 (d, 2H, J=16.0 Hz), 2.53 (s, 3H), 2.92-2.98 (m, 2H), 3.33-3.35 (m, 1H), 4.23 (d, 2H, J=16.0 Hz), 7.21-7.25 (m, 4H), 7.34 (d, 2H, J=8.0 Hz), 7.69 (d, 2H, J=8.0 Hz);

ESI-MS (m/z): 254 (M⁺+1, free amine)

The following intermediate was prepared by following the preparation of 1-[{N-(4-chlorophenyl)}aminocarbonyl]-4-aminopiperidine (pTSA salt) with the use of appropriate amine [4-amino-1-(tert-butyloxycarbonyl)piperidine] and electrophile [4-chlorophenyl isocyanate].
4-N-({4-Chlorophenyl}aminocarbonyl)-1-piperidine (pTSA salt)

[ESI-MS (m/z): 239.12 (M⁺+1), free amine];

### Synthesis of (S)-3-(4-cyanobenzyl)oxy-1-pyrrolidine (pTSA Salt)

### a. Step a: Synthesis of (S)—N-(tert-butylcarbonyloxy)-3-(4-cyanobenzyl)oxy-1-pyrrolidine

A solution of (S)—N-(tert-butylcarbonyloxy)-3-hydroxy-1-pyrrolidine (500 mg, 2.70 mmol) in anhydrous THF (2.0 mL) was added drop wise to a slurry of sodium hydride (60% dispersion in oil, 128 mg, 3.21 mmol) in THF (6.0 mL) at 0° C. under nitrogen atmosphere and the mixture stirred for 0.3 hours at 0° C. A solution of 4-cyanobenzyl bromide (576 mg, 2.94 mmol) in THF (3 mL) was added and the mixture warmed to room temperature and stirred for 18 hours. Water (20.0 mL) was added. The mixture was extracted with ethyl acetate (50.0 mL). The organic extract was washed with brine, dried over anhydrous sodium sulphate, and evaporated in vacuo. The crude product was chromatographed on silica gel (100-200 mesh) by eluting with 10% ethyl acetate in hexane to afford the colourless solid (500.0 mg, 76%)

¹H NMR (400 MHz, CDCl₃): δ 1.48 (s, 9H), 1.82-2.19 (m, 2H), 3.35-3.57 (m, 4H), 4.15-4.25 (m, 1H), 4.50-4.65 (m, 2H), 7.45 (d, J=8.1 Hz, 2H), 7.66 (d, J=8.1 Hz, 2H)

### b. Step b: Synthesis of (S)-3-(4-cyanobenzyl)oxy-1-pyrrolidine (pTSA Salt)

To a solution of the compound (600 mg, 1.99 mmol), obtained above, in acetonitrile (15.0 mL) was added pTSA (567 mg, 2.98 mmol) at ambient temperature. The mixture was stirred for 12 hours at room temperature. The solvent was evaporated and the residue taken in ethyl acetate. The mixture was stirred for 30 minutes, and the precipitated solid filtered, washed with cold ethyl acetate and dried to yield the title compound (642.0 mg, 86%).

¹H NMR (300 MHz, MeOH-d4): δ 2.0-2.20 (m, 2H), 2.36 (s, 3H), 3.32-3.53 (m, 4H), 4.30-4.40 (m, 1H), 4.64 (s, 2H), 7.23 (d, J=8.1 Hz, 2H), 7.54 (d, J=8.1 Hz, 2H), 7.69-7.74 (m, 4H);

ESI-MS (m/z): 203.16 (M⁺+1, free amine).

The following intermediates were prepared by following the preparation of (S)-3-(4-cyanobenzyl)oxy-1-pyrrolidine (pTSA salt) from appropriate amine {(S)—N-(tert-butylcarbonyloxy)-3-hydroxy-1-pyrrolidine or (R)—N-(tert-butylcarbonyloxy)-3-hydroxy-1-pyrrolidine} and appropriate electrophile, by following the preparation of (S)-3-(4-cyanobenzyl)oxy-1-pyrrolidine (pTSA salt). In those cases, where the solid didn't precipitate (semi-solid) in step b, the solvent was decanted. Fresh ethyl acetate was added and, after stirring for 5 minutes, the solvent was decanted and the resulting semi-solid was dried under vacuum to afford the pure product.
(S)-3-(4-Fluorobenzyl)oxy-1-pyrrolidine (pTSA salt)

[ESI-MS (m/z): 196.13 (M⁺+1), free amine];
(R)-3-(4-Cyanobenzyl)oxy-1-pyrrolidine (pTSA salt)

[ESI-MS (m/z): 203.13 (M⁺+1), free amine]; and
(S)-3-(Ethoxycarbonyl)methyloxy-1-pyrrolidine (pTSA salt)

[ESI-MS (m/z): 174.09 (M⁺+1), free amine].

### Synthesis of 2-(trifluoroacetyl)-2,5-diazabicyclo[2.2.1]heptane (p TSA Salt)

### a. Step a: Synthesis of 2-(trifluoroacetyl)-2,5-diazabicyclo[2.2.1]heptane

Trifluoroacetic anhydride (0.9 mL, 6.55 mmol) was added dropwise to a solution of tert-butyl-2,5-diazabicyclo[2.2.1]heptane-2-carboxylate (1.0 g, 5.04 mmol) and triethylamine (2.2 mL, 15.1 mmol) in dichloromethane (5 mL) at 0° C. over a period of 30 minutes. The mixture was stirred at room temperature for about 2-3 hours and then partitioned between water and dichloromethane. The aqueous layer was extracted with dichloromethane, the combined organic layer was washed with brine, dried over anhydrous sodium sulfate and concentrated in vacuo to afford the title product (1.30 g, % yield: 87.2%)

¹H NMR (400 MHz, MeOH-d4): δ 1.47 (s, 9H), 1.90-2.10 (m, 2H), 3.32-3.50 (m, 3H), 3.60-3.80 (m, 1H), 4.58 (brs, 1H), 4.82 (brs, 1H);

[ESI-MS (m/z): 295 (M⁺+1)].

### b. Step b: Synthesis of 2-(trifluoroacetyl)-2,5-diazabicyclo[2.2.1]heptane (p TSA Salt)

p Toluenesulfonic acid (1.26 g, 6.63 mmol) was added to a solution of the compound (1.3 g, 4.4 mmol) obtained from above step in acetonitrile (20 mL) and this reaction mixture was stirred for 12 h at room temperature. The solvent was evaporated and the residue was dissolved in ethyl acetate. The mixture was again stirred for 30 minutes and the precipitated solid was filtered, washed with cold ethyl acetate and dried to afford the title compound (1.402 g, % yield: 87.1% (as salt))

¹H NMR (400 MHz, MeOH-d4): δ 2.05-2.35 (m, 2H), 2.37 (s, 3H), 3.34-3.48 (m, 2H), 3.65-3.75 (m, 1H), 3.90 (s, 1H), 4.56 (s, 1H), 7.24 (d, J=8.0 Hz, 2H), 7.43 (d, J=8.0 Hz, 2H);

[ESI-MS (m/z): 195.2 (M⁺+1), free amine].

### Example 1

### Synthesis of (3R)—N-[1-{morpholin-1-carbonyl}piperidin-4-yl]-3-amino-4-[2,4,5-trifluoro phenyl]butanamide (TFA Salt) (Compound No. 01)

### Step a: (3R)—N-[1-{morpholin-1-carbonyl}piperidin-4-yl]-3-(n-tert-butyloxycarbonyl)amino-4-[2,4,5-trifluorophenyl] butanamide

To a mixture of 4-ammonium-1-(morpholin-1-carbonyl)piperidine 4-toluenesulphonate (84 mg, 0.21 mmol), (3R)-3-[(N-tert-butoxycarbonyl)amino]-4-(2,4,5-trifluorophenyl)butanoic acid (70 mg, 0.21 mmol), triethylamine (0.043 mL, 0.32 mmol) and 1-hydroxybenzotriazole (0.040 g, 0.26 mmol) in dichloromethane (4.0 mL) at 0° C. under N₂ atmosphere, was added EDCI (0.059 g, 0.31 mmol). The reaction mixture was stirred at 0° C. for 30 minutes and then overnight at room temperature. The solvent was evaporated and the residue partitioned between ethyl acetate and water. The organic layer was washed with aqueous citric acid (10%), water, saturated aqueous sodium bicarbonate, water and brine. The organic layer was dried over anhydrous sodium sulphate, and concentrated under reduced pressure. The residue obtained, was purified by column chromatography using 10% methanol in dichloromethane (silica gel 100-200 mesh) as eluent to yield the title compound (88 mg, 79%).

¹H NMR (400 MHz, CDCl₃): δ 1.37 (s, 9H), 1.92 (t, 2H, J=12.0 Hz), 2.25-2.53 (m, 2H), 2.75-2.95 (m, 4H), 3.24 (t, 4H, J=4.4 Hz), 3.67 (t, 6H, J=4.4 Hz), 3.82-4.1 (m, 2H), 5.36 (br d, 1H, J=8.0 Hz), 5.7 (br s, 1H), 6.80-6.92 (m, 1H), 7.00-7.10 (m, 1H);

ESI-MS (m/z): 529 (M⁺+1).

### Step b: (3R)—N-[1-{morpholin-1-carbonyl}piperidin-4-yl]-3-amino-4-[2,4,5-trifluoro phenyl] butanamide (TFA Salt)

To a solution of compound (80 mg, 0.15 mmol) in dichloromethane (2.0 mL), obtained above, at 0° C. under N₂ atmosphere, a solution of trifluoroacetic acid (5.0 mL) in dichloromethane (15.0 mL) was added dropwise. The resulting mixture was stirred at room temperature for 3 hours. The solvent was evaporated and the residue washed with diethyl ether to obtain colourless solid (53.0 mg, 64%).

¹H NMR (400 MHz, MeOH-d4): δ 1.3-1.5 (m, 2H), 2.40-2.60 (m, 2H), 2.84-3.10 (m, 4H), 3.2-3.35 (m, 8H), 3.6-3.7 (m, 6H), 3.72-3.9 (m, 2H), 7.15-7.35 (m, 2H);

ESI-MS (m/z): 429.3 (M⁺+1, free amine).

The following compounds were prepared as per the procedures given in Example 1 by coupling appropriate amines {4-amino-1-(substituted)piperidine, 4-(N-substituted)amino piperidine, 3-(O-substituted)oxypyrrolidine, 3-(N-substituted)azabicyclo[3.1.0]hexan-6-amine, 2-(N-substituted)2,5-diazabicyclo[2.2.1]heptane} with (3R)-3-[(N-tert-butoxycarbonyl)amino]-4-(2,4,5-trifluorophenyl) butanoic acid and using appropriate acid (e.g., 4-methylbenzenesulfonic acid, trifluoroacetic acid, methanolic-HCl) for deprotection. Respective free amines of the salt were prepared by taking the compound in ethyl acetate, and neutralization was carried out with 10% sodium bicarbonate.
Compound No. 2: (3R)-3-Amino-N-{1-[(4-fluorophenyl)sulphonyl]piperidin-4-yl}-4-(2,4,5-trifluorophenyl)butanamide and its 4-methylbenzenesulfonic acid salt

[ESI-MS (m/z): 474.2 (M⁺+1, free amine)];
Compound No. 3: (3R)-3-Amino-N-[1-(4-fluorobenzoyl)piperidin-4-yl]-4-(2,4,5-trifluorophenyl) butanamide and its 4-methylbenzenesulfonic acid salt

[ESI-MS (m/z): 438.1 (M⁺+1, free amine)];
Compound No. 4: N-{1-[(3R)-3-Amino-4-(2,4,5-trifluorophenyl)butanoyl]piperidin-4-yl}-4-fluorobenzamide and its trifluoroacetic acid salt

¹H NMR (400 MHz, MeOH-d4): δ 1.40-1.60 (m, 2H), 1.80-2.10 (m, 2H), 2.66-2.90 (m, 3H), 3.08 (d, 2H, J=4.0 Hz), 3.16-3.28 (m, 1H), 3.80-3.95 (m, 2H), 4.05-4.18 (m, 1H), 4.51 (d, 1H, J=12.8 Hz), 7.15-7.40 (m, 4H), 7.80-7.92 (m, 2H);
Compound No. 5: (3R)-3-Amino-N-[1-(methylsulphonyl)piperidin-4-yl]-4-(2,4,5-trifluoro phenyl)butanamide and its 4-methylbenzenesulfonic acid

[ESI-MS (m/z): 393.91 (M⁺+1, free amine)];
Compound No. 6: (3R)-3-Amino-N-(3-hydroxy-1-adamantyl)-4-(2,4,5-trifluorophenyl) butanamide and its 4-methylbenzenesulfonic acid salt

[ESI-MS (m/z): 382.91 (M⁺+1, free amine)];
Compound No. 7: 4-{[(3R)-3-Amino-4-(2,4,5-trifluorophenyl)butanoyl]amino}-N-(4-chloro phenyl)piperidine-1-carboxamide and its hydrochloride salt

[ESI-MS (m/z): 468.96 (M⁺+1, free amine)];
Compound No. 8: (3R)-3-Amino-N-[(1R,5S)-3-(2-thienylsulphonyl)-3-azabicyclo[3.1.0]hex-6-yl]-4-(2,4,5-trifluorophenyl)butanamide and its trifluoroacetic acid salt

[ESI-MS (m/z): 457.87 (M⁺+1, free amine)];
Compound No. 9: (3R)-3-Amino-N-[(1R,5S)-3-(4-cyanobenzoyl)-3-azabicyclo[3.1.0]hex-6-yl]-4-(2,4,5-trifluorophenyl)butanamide and its trifluoroacetic acid salt

[ESI-MS (m/z): 442.97 (M⁺+1, free amine)];
Compound No. 10: (3R)-3-Amino-N-[(1R,5S)-3-(2,6-difluorobenzoyl)-3-azabicyclo[3.1.0]hex-6-yl]-4-(2,4,5-trifluorophenyl)butanamide and its trifluoroacetic acid salt

[ESI-MS (m/z): 453.96 (M⁺+1, free amine)];
Compound No. 11: N-{1-[(3R)-3-Amino-4-(2,4,5-trifluorophenyl)butanoyl]piperidin-4-yl}-4-fluorobenzenesulfonamide and its trifluoroacetic acid salt

[ESI-MS (m/z): 473.89 (M⁺+1, free amine)];
Compound No. 12: N-{1-[(3R)-3-Amino-4-(2,4,5-trifluorophenyl)butanoyl]piperidin-4-yl}morpholine-4-carboxamide and its trifluoroacetic acid salt

[ESI-MS (m/z): 428.95 (M⁺+1, free amine)];
Compound No. 13: 1-{1-[(3R)-3-Amino-4-(2,4,5-trifluorophenyl)butanoyl]piperidin-4-yl}-3-(4-chlorophenyl)urea and its trifluoroacetic acid salt

[ESI-MS (m/z): 470.79 (M⁺+1, free amine)];
Compound No. 14: N-{1-[(3R)-3-Amino-4-(2,4,5-trifluorophenyl)butanoyl]piperidin-4-yl}-3-fluoro-4-methoxybenzamide and its trifluoroacetic acid salt

[ESI-MS (m/z): 467.91 (M⁺+1, free amine)];
Compound No. 15: N-{1-[(3R)-3-Amino-4-(2,4,5-trifluorophenyl)butanoyl]piperidin-4-yl}-2-propanesulfonamide and its trifluoroacetic acid salt

[ESI-MS (m/z): 421.93 (M⁺+1, free amine)];
Compound No 16: (3R)-3-Amino-N-[(1R,5S)-3-(4-trifluorobenzenesulphonyl)-3-azabicyclo [3.1.0]hex-6-yl]-4-(2,4,5-trifluorophenyl)butanamide and its trifluoroacetic acid salt

[ESI-MS (m/z): 521.71 (M⁺+1, free amine)];
Compound No. 17: (3R)-3-Amino-N-[(1R,5S)-3-(thiophene-2-carbonyl)-3-azabicyclo[3.1.0]hex-6-yl]-4-(2,4,5-trifluorophenyl)butanamide and its trifluoroacetic acid salt

[ESI-MS (m/z): 423.78 (M⁺+1, free amine)];
Compound No. 18: (3R)-3-Amino-N-[(1R,5S)-3-(ethanesulphonyl)-3-azabicyclo[3.1.0]hex-6-yl]-4-(2,4,5-trifluorophenyl)butanamide and its trifluoroacetic acid salt

[ESI-MS (m/z): 405.79 (M⁺+1, free amine)];
Compound No. 19: (3R)-3-Amino-N-[(1R,5S)-3-(4-methylbenznesulphonyl)-3-azabicyclo[3.1.0] hex-6-yl]-4-(2,4,5-trifluorophenyl)butanamide and its trifluoroacetic acid salt

[ESI-MS (m/z): 467.81 (M⁺+1, free amine)];
Compound No. 20: 4-[({(3R)-1-[(3R)-3-Amino-4-(2,4,5-trifluorophenyl)butanoyl]pyrrolidin-3-yl}oxy)methyl]benzonitrile and its trifluoroacetic acid salt

[ESI-MS (m/z): 417.96 (M⁺+1, free amine)];
Compound No. 21: (2R)-4-{(3S)-3-[(4-Fluorobenzyl)oxy]pyrrolidin-1-yl}-4-oxo-1-(2,4,5-trifluorophenyl)butan-2-amine and its trifluoroacetic acid salt

[ESI-MS (m/z): 410.84 (M⁺+1, free amine)];
Compound No. 22: 4-[({(3S)-1-[(3R)-3-Amino-4-(2,4,5-trifluorophenyl)butanoyl]pyrrolidin-3-yl}oxy)methyl]benzonitrile and its trifluoroacetic acid salt

[ESI-MS (m/z): 417.84 (M⁺+1, free amine)];
Compound No. 23: N-{1-[(3R)-3-amino-4-(2,4,5-trifluorophenyl)butanoyl]piperidin-4-yl}-2,2,2-trifluoroethanesulfonamide and its trifluoroacetic acid salt

[ESI-MS (m/z): 462.04 (M⁺+1, free amine)];
Compound No. 24: N-{1-[(3R)-3-amino-4-(2,4,5-trifluorophenyl)butanoyl]piperidin-4-yl}-4-cyanobenzenesulfonamide and its trifluoroacetic acid salt

[ESI-MS (m/z): 480.78 (M⁺+1, free amine)];
Compound No. 25: N-{1-[3-amino-4-(2,4,5-trifluorophenyl)butanoyl]piperidin-4-yl}-2,4-difluorobenzenesulfonamide and its trifluoroacetic acid salt

[ESI-MS (m/z): 491.79 (M⁺+1, free amine)];
Compound No. 26: Methyl 5-[({1-[(3R)-3-amino-4-(2,4,5-trifluorophenyl)butanoyl]piperidin-4-yl} amino) sulfonyl]-4-methylthiophene-2-carboxylate

[ESI-MS (m/z): 534.10 (M⁺+1, (m/z)];
Compound No. 27: N-{1-[(3R)-3-amino-4-(2,4,5-trifluorophenyl)butanoyl]piperidin-4-yl}-1,3,5-trimethyl-1H-pyrazole-4-sulfonamide

[ESI-MS (m/z): 488.10 (M⁺+1, (m/z)];
Compound No. 28: methyl 4-[({1-[(3R)-3-amino-4-(2,4,5-trifluorophenyl)butanoyl]piperidin-4-yl} amino)sulfonyl]-2,5-dimethyl-3-furoate

[ESI-MS (m/z): 532.18 (M⁺+1, (m/z)];
Compound No. 29: N-{1-[(3R)-3-amino-4-(2,4,5-trifluorophenyl)butanoyl]piperidin-4-yl}-4-fluorobenzenesulfonamide

[ESI-MS (m/z): 474.14 (M⁺+1, (m/z)];
Compound No. 30: 4-acetyl-N-{1-[(3R)-3-amino-4-(2,4,5-trifluorophenyl)butanoyl] piperidin-4-yl}benzenesulfonamide and its trifluoroacetic acid salt

[ESI-MS (m/z): 532.18 (M⁺+1, free amine)];
Compound No. 31: N-{1-[(3R)-3-amino-4-(2,4,5-trifluorophenyl)butanoyl]piperidin-4-yl}-2,4-difluorobenzenesulfonamide and its trifluoroacetic acid salt

[ESI-MS (m/z): 492 (M⁺+1, free amine)];
Compound No. 32: N-{1-[(3R)-3-amino-4-(2,4,5-trifluorophenyl)butanoyl]piperidin-4-yl} methane sulfonamide and its trifluoroacetic acid salt

[ESI-MS (m/z): 394 (M⁺+1, free amine)];
Compound No. 33: methyl 5-[({1-[(3R)-3-amino-4-(2,4,5-trifluorophenyl)butanoyl] piperidin-4-yl} amino) sulfonyl]-4-methylthiophene-2-carboxylate and its trifluoroacetic acid salt

[ESI-MS (m/z): 534 (M⁺+1, free amine)];
Compound No. 34: N-{1-[(3R)-3-amino-4-(2,4,5-trifluorophenyl)butanoyl]piperidin-4-yl} propane-2-sulfonamide and its trifluoroacetic acid salt

[ESI-MS (m/z): 422 (M⁺+1, free amine)];
Compound No. 35: N-{(3S)-1-[(3R)-3-amino-4-(2,4,5-trifluorophenyl)butanoyl]piperidin-3-yl}ethane sulfonamide and its trifluoroacetic acid salt

[ESI-MS (m/z): 408 (M⁺+1, free amine)];
Compound No. 36: 4-(1,3-dihydro-2H-isoindol-2-yl)-4-oxo-1-(2,4,5-trifluorophenyl)butan-2-amine and its trifluoroacetic acid salt

[ESI-MS (m/z): 335.10 (M⁺+1, free amine)];
Compound No. 37: N-{1-[3-amino-4-(2,4,5-trifluorophenyl)butanoyl]piperidin-4-yl} propanamide and its trifluoroacetic acid salt

[ESI-MS (m/z): 372.10 (M⁺+1, free amine)];
Compound No. 38: N-{1-[(3R)-3-amino-4-(2,4,5-trifluorophenyl)butanoyl]piperidin-4-yl}acetamide and its trifluoroacetic acid salt

[ESI-MS (m/z): 358.13 (M⁺+1, free amine)];
Compound No. 39: N-{1-[(3R)-3-amino-4-(2,4,5-trifluorophenyl)butanoyl]piperidin-4-yl}-4-fluorobenzamide

[ESI-MS (m/z): 438.19 (M⁺+1, (m/z)];
Compound No. 40: 2-({1-[(3R)-3-amino-4-(2,4,5-trifluorophenyl)butanoyl]piperidin-4-yl} amino)-4,6-difluorobenzonitrile and its trifluoroacetic acid salt

[ESI-MS (m/z): 453.22 (M⁺+1, free amine)];
Compound No. 41: 2-({1-[(3R)-3-amino-4-(2,4,5-trifluorophenyl)butanoyl]piperidin-4-yl} amino)benzonitrile and its trifluoroacetic acid salt

[ESI-MS (m/z): 417.20 (M⁺+1, free amine)];
Compound No. 42: 4-({1-[(3R)-3-amino-4-(2,4,5-trifluorophenyl)butanoyl]piperidin-4-yl} amino)-2,6-difluorobenzonitrile and its 4-methylbenzenesulfonic acid salt

[ESI-MS (m/z): 453.22 (M⁺+1, free amine)];
Compound No. 43: 4-({1-[(3R)-3-amino-4-(2,4,5-trifluorophenyl)butanoyl]piperidin-4-yl} amino)-2,6-difluorobenzonitrile and its trifluoroacetic acid salt

[ESI-MS (m/z): 452 (M⁺+1, free amine)];
Compound No. 44: 2-({1-[(3R)-3-amino-4-(2,4,5-trifluorophenyl)butanoyl]piperidin-4-yl} amino)benzonitrile and its trifluoroacetic acid salt

[ESI-MS (m/z): 417.29 (M⁺+1, free amine)];
Compound No. 45: 4-({1-[(3R)-3-amino-4-(2,4,5-trifluorophenyl)butanoyl]piperidin-4-yl} amino)benzonitrile and its trifluoroacetic acid salt

[ESI-MS (m/z): 417.20 (M⁺+1, free amine)];
Compound No. 46: 4-({1-[(3R)-3-amino-4-(2,4,5-trifluorophenyl)butanoyl]piperidin-4-yl} amino)benzonitrile and its trifluoroacetic acid salt

[ESI-MS (m/z): 417 (M⁺+1, free amine)];
Compound No. 47: 4-({1-[(3R)-3-amino-4-(2,4,5-trifluorophenyl)butanoyl]piperidin-4-yl} amino)-2-(trifluoromethyl)benzonitrile and its trifluoroacetic acid salt

[ESI-MS (m/z): 485 (M⁺+1, free amine)];
Compound No. 48: Ethyl ({(3S)-1-[(3R)-3-amino-4-(2,4,5-trifluorophenyl)butanoyl]pyrrolidin-3-yl}oxy)acetate and its trifluoroacetic acid salt

[ESI-MS (m/z): 389.27 (M⁺+1, free amine)];
Compound No. 53: (2R)-4-(5-acetyl-2,5-diazabicyclo[2.2.1]hept-2-yl)-4-oxo-1-(2,4,5-trifluoro-phenyl)butan-2-amine and its trifluoroacetic acid salt

[ESI-MS (m/z): 356.31 (M⁺+1, free amine)];
Compound No. 60: (2R)-4-{5-[(3-fluorophenyl)sulfonyl]-2,5-diazabicyclo[2.2.1]hept-2-yl}-4-oxo-1-(2,4,5-trifluorophenyl)butan-2-amine and its trifluoroacetic acid salt

[ESI-MS (m/z): 472 (M⁺+1, free amine)];
Compound No. 71: (2R)-4-[5-(ethylsulfonyl)-2,5-diazabicyclo[2.2.1]hept-2-yl]-4-oxo-1-(2,4,5-trifluorophenyl)butan-2-amine and its trifluoroacetic acid salt

[ESI-MS (m/z): 406.24 (M⁺+1, free amine)];
Compound No. 74: 4-{5-[(3R)-3-amino-4-(2,4,5-trifluorophenyl)butanoyl]-2,5-diazabicyclo-[2.2.1]hept-2-yl}-2-(trifluoromethyl)benzonitrile and its trifluoroacetic acid salt

[ESI-MS (m/z): 483.12 (M⁺+1, free amine)];
Compound No. 76: (2R)-4-[5-(4-fluorobenzoyl)-2,5-diazabicyclo[2.2.1]hept-2-yl]-4-oxo-1-(2,4,5-trifluorophenyl)butan-2-amine and its trifluoroacetic acid salt

[ESI-MS (m/z): 436.17 (M⁺+1, free amine)];
Compound No. 78: 5-[3-amino-4-(2,4,5-trifluorophenyl)butanoyl]-N-(4-fluorophenyl)-2,5-diaza-bicyclo[2.2.1]heptane-2-carboxamide and its trifluoroacetic acid salt

[ESI-MS (m/z): 451.25 (M⁺+1, free amine)];
Compound No. 81: (2R)-4-oxo-4-[5-(2-thienylsulfonyl)-2,5-diaza-bicyclo[2.2.1]hept-2-yl]-1-(2,4,5-trifluorophenyl)butan-2-amine and its trifluoroacetic acid salt

[ESI-MS (m/z): 460 (M⁺+1, free amine)];
Compound No. 83: 4-({5-[(3R)-3-amino-4-(2,4,5-trifluorophenyl)butanoyl]-2,5-diazabicyclo [2.2.1]hept-2-yl}sulfonyl)benzonitrile and its trifluoroacetic acid salt

[ESI-MS (m/z): 479 (M⁺+1, free amine)];
Compound No. 85: (2R)-4-{(1S,4S)-5-[(3,5-difluorophenyl)sulfonyl]-2,5-diazabicyclo [2.2.1]hept-2-yl}-4-oxo-1-(2,4,5-trifluorophenyl) butan-2-amine and its trifluoroacetic acid salt

[ESI-MS (m/z): 490 (M⁺+1, free amine)];
Compound No. 90: (2R)-4-oxo-4-[5-(propylsulfonyl)-2,5-diazabicyclo[2.2.1]hept-2-yl]-1-(2,4,5-trifluorophenyl)butan-2-amine and its trifluoroacetic acid salt

[ESI-MS (m/z): 420.26 (M⁺+1, free amine)];
Compound No. 91: (2R)-4-[5-(isopropylsulfonyl)-2,5-diazabicyclo[2.2.1]hept-2-yl]-4-oxo-1-(2,4,5-trifluorophenyl)butan-2-amine and its trifluoroacetic acid salt

[ESI-MS (m/z): 420.40 (M⁺+1, free amine)];
Compound No. 92: (2R)-4-[5-(butylsulfonyl)-2,5-diazabicyclo[2.2.1]hept-2-yl]-4-oxo-1-(2,4,5-trifluorophenyl)butan-2-amine and its trifluoroacetic acid salt

[ESI-MS (m/z): 434.30 (M⁺+1, free amine)];
Compound No. 93: (2R)-4-oxo-4-[5-(phenylsulfonyl)-2,5-diazabicyclo[2.2.1]hept-2-yl]-1-(2,4,5-trifluorophenyl)butan-2-amine and its trifluoroacetic acid salt

[ESI-MS (m/z): 454.13 (M⁺+1, free amine)];
Compound No. 95: (2R)-4-oxo-4-(5-{[3-(trifluoromethoxy)phenyl]sulfonyl}-2,5-diazabicyclo [2.2.1]hept-2-yl)-1-(2,4,5-trifluorophenyl)butan-2-amine and its trifluoroacetic acid salt

[ESI-MS (m/z): 538.21 (M⁺+1, free amine)]; and
Compound No. 96: (2R)-4-[5-(methylsulfonyl)-2,5-diazabicyclo[2.2.1]hept-2-yl]-4-oxo-1-(2,4,5-trifluorophenyl)butan-2-amine and its trifluoroacetic acid salt

[ESI-MS (m/z): 392 (M⁺+1, free amine)].

### Example 2

### Synthesis of (2R)-4-oxo-4-[5-(trifluoroacetyl)-2,5-diazabicyclo[2.2.1]hept-2-yl]-1-(2,4,5-trifluorophenyl)butan-2-amine (HCl Salt) (Compound No. 50)

### Step a: Synthesis of tert-butyl [(1R)-3-oxo-3-[5-(trifluoroacetyl)-2,5-diazabicyclo[2.2.1]hept-2-yl]-1-(2,4,5-trifluorobenzyl)propyl]carbamate

To a solution of 2-(trifluoroacetyl)-2,5-diazabicyclo[2.2.1]heptane (p TSA salt) (0.88 g, 2.4 mmol) and (3R)-3-[(tert-butoxycarbonyl)amino]-4-(2,4,5-trifluorophenyl)butanoic acid (0.66 g, 2.0 mmol) in dry dimethylformamide, triethylamine (0.58 mL, 4.0 mmol) and n-hydroxybenzotriazole (0.39 g, 2.4 mmol) at 0° C. for 10 minutes and then 1-ethyl-3-(3′-dimethylaminopropyl)carbodiimide (0.5 g, 2.4 mmol) was added. After the removal of ice bath, reaction was allowed to stir at ambient temperature for about 14 hours. The reaction mixture was decomposed in cold water and the product was extracted using ethyl acetate. The organic layer was dried over anhydrous sodium sulfate and concentrated over vacuo. The residue thus obtained was purified by column chromatography over silica gel (100-200 mesh) using ethyl acetate and hexane as eluents to give the title product (0.43 g, % yield: 35.5%)

¹H NMR (400 MHz, CDCl₃): δ 1.33 (s, 9H), 1.90-2.10 (m, 2H), 2.40-2.50 (m, 1H), 2.60-2.75 (m, 2H), 2.80-2.95 (m, 1H), 3.51-3.73 (m, 4H), 4.15 (brs, 1H), 4.66-4.92 (m, 1H), 7.05-7.44 (m, 2H);

ESI-MS (m/z): 510.30 (M⁺+1) (m/z).

### Step b: Synthesis of (2R)-4-oxo-4-[5-(trifluoroacetyl)-2,5-diazabicyclo[2.2.1]hept-2-yl]-1-(2,4,5-trifluorophenyl)butan-2-amine (HCl Salt)

The product obtained from the above step (0.05 g, 0.1 mmol) was dissolved in methanolic-HCl (2.5 N) and stirred for overnight at room temperature. The reaction mixture was concentrated and the residue was taken in diethyl ether, filtered and dried under vacuum to obtain the title compound (0.023 g, % yield: 57.5%)

¹H NMR (400 MHz, CDCl₃): δ 1.8-2.1 (m, 2H), 2.35-3.05 (m, 4H), 3.30-3.83 (m, 5H), 4.35-4.70 (m, 2H), 7.11-7.32 (m, 2H);

ESI-MS (m/z): 410.18 (M⁺+1) (m/z).

### Example 3

### Synthesis of (2R)-4-oxo-4-(5-propionyl-2,5-diazabicyclo[2.2.1]hept-2-yl)-1-(2,4,5-trifluoro-phenyl)butan-2-amine (HCl Salt) (Compound No. 51)

### Step a: Synthesis of tert-butyl [(1R)-3-(2,5-diazabicyclo[2.2.1]hept-2-yl)-3-oxo-1-(2,4,5-trifluorobenzyl)propyl]carbamate

To a solution of compound obtained in step a of Example 2 (0.1 g, 0.2 mmol) in methanol (2 mL) was added saturated solution of potassium carbonate (0.5 mL) at room temperature and this reaction mixture was stirred at the same temperature for overnight. The resultant mixture was concentrated and water (10 mL) was added to it. The compound was extracted out with ethyl acetate and the combined organic layers were dried over anhydrous sodium sulfate, concentrated and dried under vacuum to get the title compound (0.72 g, % yield: 87.5%)

¹H NMR (400 MHz, CD3OD): δ 1.33 (s, 9H), 1.72-1.90 (m, 2H), 2.40-2.80 (m, 3H), 2.85-3.0 (m, 2H), 3.01-3.30 (m, 1H), 3.76 (d, J=10 Hz, 1H), 4.05-4.19 (m, 1H), 4.54-4.71 (m, 1H), 7.06-7.44 (m, 2H);

ESI-MS (m/z): 414.35 (M⁺+1) (m/z).

### Step b: Synthesis of tert-butyl [(1R)-3-oxo-3-(5-propionyl-2,5-diazabicyclo[2.2.1]hept-2-yl)-1-(2,4,5-trifluorobenzyl)propyl]carbamate

To the solution of the compound as obtained in step a (0.1 g, 0.24 mmol), dry triethylamine (0.1 mL, 0.72 mmol) in dichloromethane (5 mL) was added a solution of propionyl chloride (0.03 mL, 0.32 mmol) dropwise at room temperature. The reaction mixture was stirred at same temperature for overnight and then partitioned between dichloromethane and water. The crude compound was extracted from aqueous layer using dichloromethane and combined layers were washed using brine, dried over anhydrous sodium sulfate and concentrated. The hence obtained compound was purified by column chromatography over silica gel (100-200 mesh) using ethyl acetate-hexane as eluents to get the title compound (0.72 g, % yield: 63.7%)

ESI-MS (m/z): 470 (M⁺+1) (m/z).

### Step c: Synthesis of (2R)-4-oxo-4-(5-propionyl-2,5-diazabicyclo[2.2.1]hept-2-yl)-1-(2,4,5-trifluorophenyl) butan-2-amine (HCl Salt)

The compound obtained from step b (0.50 g, 0.11 mmol) was dissolved in methanolic-HCl (2.5 N) at room temperature. The reaction mixture was stirred for overnight and then concentrated. The resultant residue was stirred in diethyl ether for 10 minutes, filtered and dried to obtain the title compound (0.215 g, % yield: 52.8%)

¹H NMR (400 MHz, MeOH-d4): δ 1.06-1.15 (m, 3H), 1.85-2.05 (m, 2H), 2.20-2.60 (m, 4H), 2.72-2.82 (m, 2H), 3.35-3.70 (m, 5H), 4.55-4.62 (m, 2H), 7.13-7.33 (m, 2H);

ESI-MS (m/z): 370.21 (M⁺+1).

The following compounds have been prepared using similar procedure using appropriate acid (e.g., 4-methylbenzenesulfonic acid, trifluoroacetic acid or methanolic-HCl) for deprotection as mentioned earlier.
Compound No. 49: (2R)-4-oxo-4-[5-(2-thienylacetyl)-2,5-diazabicyclo[2.2.1]hept-2-yl]-1-(2,4,5-trifluorophenyl)butan-2-amine and its trifluoroacetic acid salt

ESI-MS (m/z): 438.13 (M⁺+1) free amine.
Compound No. 52: 5-[(3R)-3-amino-4-(2,4,5-trifluoro phenyl)butanoyl]-N-(4-cyanophenyl)-2,5-diazabicyclo[2.2.1]heptane-2-carboxamide and its 4-methylbenzenesulfonic acid salt

ESI-MS (m/z): 458.24 (M⁺+1), free amine.
Compound No. 54: (1S,4S)-5-[(3R)-3-amino-4-(2,4,5-trifluorophenyl)butanoyl]-N-(4-fluoro-phenyl)-2,5-diazabicyclo[2.2.1]heptane-2-carboxamide and its trifluoroacetic acid salt

ESI-MS (m/z): 451.34 (M⁺+1), free amine.
Compound No. 55: (1S,4S)-5-[(3R)-3-amino-4-(2,4,5-trifluorophenyl)butanoyl]-N-(4-methoxy-phenyl)-2,5-diazabicyclo[2.2.1]heptane-2-carboxamide and its trifluoroacetic acid salt

ESI-MS (m/z): 463.30 (M⁺+1) free amine.
Compound No. 56: (1S,4S)-5-[(3R)-3-amino-4-(2,4,5-trifluorophenyl)butanoyl]-N-[4-(trifluoro-methyl)phenyl]-2,5-diazabicyclo[2.2.1]heptane-2-carboxamide and its trifluoroacetic acid salt

ESI-MS (m/z): 501.33 (M⁺+1) free amine.
Compound No. 57: (1S,4S)-5-[(3R)-3-amino-4-(2,4,5-trifluorophenyl)butanoyl]-N-benzyl-2,5-diazabicyclo[2.2.1]heptane-2-carboxamide and its trifluoroacetic acid salt

ESI-MS (m/z): 447.31 (M⁺+1) free amine.
Compound No. 58: (1S,4S)-5-[(3R)-3-amino-4-(2,4,5-trifluorophenyl)butanoyl]-N-(4-methyl-phenyl)-2,5-diazabicyclo[2.2.1]heptane-2-carboxamide and its trifluoroacetic acid salt

ESI-MS (m/z): 447.31 (M⁺+1) free amine.
Compound No. 59: (1S,4S)-5-[(3R)-3-amino-4-(2,4,5-trifluorophenyl)butanoyl]-N-tert-butyl-2,5-diaza-bicyclo[2.2.1]heptane-2-carboxamide and its trifluoroacetic acid salt

ESI-MS (m/z): 413.39 (M⁺+1) free amine.
Compound No. 61: (1S,4S)-5-[(3R)-3-amino-4-(2,4,5-trifluorophenyl)butanoyl]-N-(3-fluoro-phenyl)-2,5-diazabicyclo[2.2.1]heptane-2-carboxamide and its trifluoroacetic acid salt

ESI-MS (m/z): 451.33 (M⁺+1) free amine.
Compound No. 62: (1S,4S)-5-[(3R)-3-amino-4-(2,4,5-trifluorophenyl)butanoyl]-N-[2-(trifluoromethyl)phenyl]-2,5-diazabicyclo[2.2.1]heptane-2-carboxamide and its trifluoroacetic acid salt

ESI-MS (m/z): 501.30 (M⁺+1) free amine.
Compound No. 63: (1S,4S)-5-[(3R)-3-amino-4-(2,4,5-trifluorophenyl)butanoyl]-N-(2-methyl-phenyl)-2,5-diazabicyclo[2.2.1]heptane-2-carboxamide and its trifluoroacetic acid salt

ESI-MS (m/z): 447.37 (M⁺+1) free amine.
Compound No. 64: (1S,4S)-5-[(3R)-3-amino-4-(2,4,5-trifluorophenyl)butanoyl]-N-(4-nitro-phenyl)-2,5-diazabicyclo[2.2.1]heptane-2-carboxamide and its trifluoroacetic acid salt

ESI-MS (m/z): 478.33 (M⁺+1) free amine.
Compound No. 65: 4-{(1S,4S)-5-[(3R)-3-amino-4-(2,4,5-trifluorophenyl)butanoyl]-2,5-diaza-bicyclo[2.2.1]hept-2-yl}-2-chlorobenzonitrile and its trifluoroacetic acid salt

ESI-MS (m/z): 449.22 (M⁺+1), free amine.
Compound No. 66: 2-{(1S,4S)-5-[(3R)-3-amino-4-(2,4,5-trifluorophenyl)butanoyl]-2,5-diaza-bicyclo[2.2.1]hept-2-yl}-6-fluorobenzonitrile and its trifluoroacetic acid salt

ESI-MS (m/z): 433.32 (M⁺+1), free amine.
Compound No. 67: 4-{(1S,4S)-5-[(3R)-3-amino-4-(2,4,5-trifluorophenyl)butanoyl]-2,5-diaza-bicyclo[2.2.1]hept-2-yl}-3-fluorobenzonitrile and its trifluoroacetic acid salt

ESI-MS (m/z): 433.32 (M⁺+1), free amine.
Compound No. 68: (1S,4S)-5-[(3R)-3-amino-4-(2,4,5-trifluorophenyl)butanoyl]-N-cyclohexyl-2,5-diazabicyclo[2.2.1]heptane-2-carboxamide and its trifluoroacetic acid salt

ESI-MS (m/z): 439.10 (M⁺+1), free amine.
Compound No. 69: (1S,4S)-5-[(3R)-3-amino-4-(2,4,5-trifluorophenyl)butanoyl]-N-(3-methoxy-phenyl)-2,5-diazabicyclo[2.2.1]heptane-2-carboxamide and its trifluoroacetic acid salt

ESI-MS (m/z): 463.07 (M⁺+1), free amine.
Compound No. 70: (1S,4S)-5-[(3R)-3-amino-4-(2,4,5-trifluorophenyl)butanoyl]-N-(2-fluoro-phenyl)-2,5-diazabicyclo[2.2.1]heptane-2-carboxamide and its trifluoroacetic acid salt

ESI-MS (m/z): 451.02 (M⁺+1), free amine.
Compound No. 72: (1S,4S)-5-[(3R)-3-amino-4-(2,4,5-trifluorophenyl)butanoyl]-N-(2,4-dimethoxyphenyl)-2,5-diazabicyclo[2.2.1]heptane-2-carboxamide and its trifluoroacetic acid salt

ESI-MS (m/z): 493.06 (M⁺+1), free amine.
Compound No. 73: (1S,4S)-5-[(3R)-3-amino-4-(2,4,5-trifluorophenyl)butanoyl]-N-isopropyl-2,5-diazabicyclo[2.2.1]heptane-2-carboxamide and its trifluoroacetic acid salt

ESI-MS (m/z): 399.08 (M⁺+1), free amine.
Compound No. 75: (1S,4S)-5-[(3R)-3-amino-4-(2,4,5-trifluorophenyl)butanoyl]-N-[4-(benzyl-oxy)phenyl]-2,5-diazabicyclo[2.2.1]heptane-2-carboxamide and its trifluoroacetic acid salt

ESI-MS (m/z): 555.06 (M⁺+17), free amine.
Compound No. 77: (1S,4S)-5-[(3R)-3-amino-4-(2,4,5-trifluorophenyl)butanoyl]-N-(3,4,5-tri-methoxyphenyl)-2,5-diazabicyclo[2.2.1]heptane-2-carboxamide and its trifluoroacetic acid salt

ESI-MS (m/z): 523.06 (M⁺+1), free amine.
Compound No. 79: (1S,4S)-5-[3-amino-4-(2,4,5-trifluorophenyl)butanoyl]-N-(2,6-diisopropyl phenyl)-2,5-diazabicyclo[2.2.1]heptane-2-carboxamide and its trifluoroacetic acid salt

ESI-MS (m/z): 517.10 (M⁺+1), free amine.
Compound No. 80: methyl 2-[({(1S,4S)-5-[(3R)-3-amino-4-(2,4,5-trifluorophenyl)butanoyl]-2,5-diaza-bicyclo[2.2.1]hept-2-yl}carbonyl)amino]benzoate and its trifluoroacetic acid salt

ESI-MS (m/z): 491.05 (M⁺+1), free amine.
Compound No. 82: (1S,4S)-5-[(3R)-3-amino-4-(2,4,5-trifluorophenyl)butanoyl]-N-(5-chloro-2-methoxy phenyl)-2,5-diazabicyclo [2.2.1]heptane-2-carboxamide and its trifluoroacetic acid salt

ESI-MS (m/z): 497.01 (M⁺+1), free amine.
Compound No. 84: (1S,4S)-5-[(3R)-3-amino-4-(2,4,5-trifluorophenyl)butanoyl]-N-(2,3-dichlorophenyl)-2,5-diazabicyclo [2.2.1]heptane-2-carboxamide and its trifluoroacetic acid salt

ESI-MS (m/z): 500.90 (M⁺+1), free amine.
Compound No. 86: (1S,4S)—N-(4-acetylphenyl)-5-[(3R)-3-amino-4-(2,4,5-trifluorophenyl)-butanoyl]-2,5-diazabicyclo[2.2.1]heptane-2-carboxamide and its trifluoroacetic acid salt

ESI-MS (m/z): 475.05 (M⁺+1), free amine.
Compound No. 87: methyl 5-[(3R)-3-amino-4-(2,4,5-trifluorophenyl)butanoyl]-2,5-diazabicyclo[2.2.1]heptane-2-carboxylate and its trifluoroacetic acid salt

ESI-MS (m/z): 372 (M⁺+1) free amine.
Compound No. 88: (1S,4S)-5-[(3R)-3-amino-4-(2,4,5-trifluorophenyl)butanoyl]-N-(2,5-dimethoxyphenyl)-2,5-diazabicyclo[2.2.1]heptane-2-carboxamide and its trifluoroacetic acid salt

ESI-MS (m/z): 493.06 (M⁺+1), free amine
Compound No. 89: ethyl 5-[(3R)-3-amino-4-(2,4,5-trifluorophenyl)butanoyl]-2,5-diazabicyclo [2.2.1]heptane-2-carboxylate and its trifluoroacetic acid salt

ESI-MS (m/z): 386 (M⁺+1) free amine.
Compound No. 94: (2R)-4-[5-(morpholin-4-ylcarbonyl)-2,5-diazabicyclo[2.2.1]hept-2-yl]-4-oxo-1-(2,4,5-trifluorophenyl)butan-2-amine and its trifluoroacetic acid salt

ESI-MS (m/z): 427.35 (M⁺+1) free amine.
Compound No. 97: (1S,4S)-5-[(3R)-3-amino-4-(2,4,5-trifluorophenyl)butanoyl]-N-(2,6-difluoro-phenyl)-2,5-diazabicyclo [2.2.1]heptane-2-carboxamide and its trifluoroacetic acid salt

ESI-MS (m/z): 469.03 (M⁺+1), free amine.
Compound No. 98: (1S,4S)-5-[(3R)-3-amino-4-(2,4,5-trifluorophenyl)butanoyl]-N-(2,4,6-trifluoro-phenyl)-2,5-diazabicyclo[2.2.1]heptane-2-carboxamide and its trifluoroacetic acid salt

ESI-MS (m/z): 487.04 (M⁺+1), free amine.
Compound No. 99: (1S,4S)—N-(3-acetylphenyl)-5-[(3R)-3-amino-4-(2,4,5-trifluorophenyl) butanoyl]-2,5-diazabicyclo[2.2.1]heptane-2-carboxamide and its trifluoroacetic acid salt

ESI-MS (m/z): 475.05 (M⁺+1), free amine.
Compound No. 100: (1S,4S)-5-[(3R)-3-amino-4-(2,4,5-trifluorophenyl)butanoyl]-N-(2,5-dichloro-phenyl)-2,5-diazabicyclo[2.2.1]heptane-2-carboxamide and its trifluoroacetic acid salt

ESI-MS (m/z): 500.97 (M⁺+1), free amine.
Compound No. 101: (1S,4S)-5-[(3R)-3-amino-4-(2,4,5-trifluorophenyl)butanoyl]-N-(4-isopropyl-phenyl)-2,5-diazabicyclo[2.2.1]heptane-2-carboxamide and its trifluoroacetic acid salt

ESI-MS (m/z): 475.05 (M⁺+1) free amine.
Compound No. 102: (1S,4S)-5-[(3R)-3-amino-4-(2,4,5-trifluorophenyl)butanoyl]-N-(4-butyl-phenyl)-2,5-diazabicyclo[2.2.1]heptane-2-carboxamide and its trifluoroacetic acid salt

ESI-MS (m/z): 489.08 (M⁺+1) free amine.
Compound No. 103: (1S,4S)-5-[(3R)-3-amino-4-(2,4,5-trifluorophenyl)butanoyl]-N-(4-ethoxy-phenyl)-2,5-diazabicyclo[2.2.1]heptane-2-carboxamide and its trifluoroacetic acid salt

ESI-MS (m/z): 477.07 (M⁺+1) free amine.
Compound No. 104: (1S,4S)-5-[(3R)-3-amino-4-(2,4,5-trifluorophenyl)butanoyl]-N-(3-ethyl-phenyl)-2,5-diazabicyclo[2.2.1]heptane-2-carboxamide and its trifluoroacetic acid salt

ESI-MS (m/z): 461.06 (M⁺+1) free amine.
Compound No. 105: (1S,4S)-5-[(3R)-3-amino-4-(2,4,5-trifluorophenyl)butanoyl]-N-(2-isopropyl-6-methylphenyl)-2,5-diazabicyclo[2.2.1]heptane-2-carboxamide and its trifluoroacetic acid salt

ESI-MS (m/z): 489.08 (M⁺+1) free amine.
Compound No. 106: (1S,4S)-5-[(3R)-3-amino-4-(2,4,5-trifluorophenyl)butanoyl]-N-mesityl-2,5-diazabicyclo[2.2.1]heptane-2-carboxamide and its trifluoroacetic acid salt

ESI-MS (m/z): 475.13 (M⁺+1) free amine.
Compound No. 107: (1S,4S)-5-[(3R)-3-amino-4-(2,4,5-trifluorophenyl)butanoyl]-N-(4-methoxy-2-methylphenyl)-2,5-diazabicyclo[2.2.1]heptane-2-carboxamide and its trifluoroacetic acid salt

ESI-MS (m/z): 477.04 (M⁺+1) free amine.
Compound No. 108: (1S,4S)-5-[(3R)-3-amino-4-(2,4,5-trifluorophenyl)butanoyl]-N-(4-phenoxy-phenyl)-2,5-diazabicyclo[2.2.1]heptane-2-carboxamide and its trifluoroacetic acid salt

ESI-MS (m/z): 525.05 (M⁺+1) free amine.
Compound No. 109: (2R)-4-[(1R,4R)-5-(cyclohexylcarbonyl)-2,5-diazabicyclo[2.2.1]hept-2-yl]-4-oxo-1-(2,4,5-trifluorophenyl) butan-2-amine and its trifluoroacetic acid salt

ESI-MS (m/z): 424.06 (M⁺+1) free amine.
Compound No. 110: (2R)-4-[(1R,4R)-5-(cyclopropyl carbonyl)-2,5-diazabicyclo [2.2.1]hept-2-yl]-4-oxo-1-(2,4,5-trifluorophenyl) butan-2-amine and its trifluoroacetic acid salt

ESI-MS (m/z): 382.06 (M⁺+1) free amine.
Compound No. 112: (1R,4R)-5-[(3R)-3-amino-4-(2,4,5-trifluorophenyl)butanoyl]-N-(2-ethoxyphenyl)-2,5-diazabicyclo[2.2.1]heptane-2-carboxamide and its trifluoroacetic acid salt

ESI-MS (m/z): 477.04 (M⁺+1) free amine.
Compound No. 113: (1R,4R)-5-[(3R)-3-amino-4-(2,4,5-trifluorophenyl)butanoyl]-N-(2-ethylphenyl)-2,5-diazabicyclo[2.2.1]heptane-2-carboxamide and its trifluoroacetic acid salt

ESI-MS (m/z): 461.05 (M⁺+1) free amine.
Compound No. 114: methyl 3-[({(1R,4R)-5-[(3R)-3-amino-4-(2,4,5-trifluorophenyl)butanoyl]-2,5-diazabicyclo[2.2.1]hept-2-yl}carbonyl)amino]benzoate and its trifluoroacetic Acid Salt

ESI-MS (m/z): 493.05 (M⁺+1) free amine.
Compound No. 115: (1R,4R)-5-[(3R)-3-amino-4-(2,4,5-trifluorophenyl)butanoyl]-N-(4-ethylphenyl)-2,5-diazabicyclo[2.2.1]heptane-2-carboxamide and its trifluoroacetic acid salt

ESI-MS (m/z): 461.05 (M⁺+1) free amine.
Compound No. 116: (1R,4R)-5-[(3R)-3-amino-4-(2,4,5-trifluorophenyl)butanoyl]-N-(3-chloro-4-methylphenyl)-2,5-diazabicyclo[2.2.1]heptane-2-carboxamide and its trifluoroacetic acid salt

ESI-MS (m/z): 480.99 (M⁺+1) free amine.
Compound No. 117: (1R,4R)-5-[(3R)-3-amino-4-(2,4,5-trifluorophenyl)butanoyl]-N-[3-(methylthio)phenyl]-2,5-diazabicyclo[2.2.1]heptane-2-carboxamide and its trifluoroacetic acid salt

ESI-MS (m/z): 479.02 (M⁺+1) free amine.
Compound No. 118: (1R,4R)-5-[(3R)-3-amino-4-(2,4,5-trifluorophenyl)butanoyl]-N-(2,4-difluorophenyl)-2,5-diazabicyclo[2.2.1]heptane-2-carboxamide and its trifluoroacetic acid salt

ESI-MS (m/z): 469.02 (M⁺+1) free amine.
Compound No. 119: (1R,4R)-5-[(3R)-3-amino-4-(2,4,5-trifluorophenyl)butanoyl]-N-(2,4-dimethylphenyl)-2,5-diazabicyclo[2.2.1]heptane-2-carboxamide and its trifluoroacetic acid salt

ESI-MS (m/z): 461.06 (M⁺+1) free amine.
Compound No. 120: (1R,4R)-5-[(3R)-3-amino-4-(2,4,5-trifluorophenyl)butanoyl]-N-(3,4-dichlorophenyl)-2,5-diazabicyclo[2.2.1]heptane-2-carboxamide and its trifluoroacetic acid salt

ESI-MS (m/z): 502.92 (M⁺+1) free amine.
Compound No. 121: (1R,4R)-5-[(3R)-3-amino-4-(2,4,5-trifluorophenyl)butanoyl]-N-[4-chloro-3-(trifluoromethyl)phenyl]-2,5-diazabicyclo[2.2.1]heptane-2-carboxamide and its trifluoroacetic acid salt

ESI-MS (m/z): 534.93 (M⁺+1) free amine.
Compound No. 122: (1R,4R)-5-[(3R)-3-amino-4-(2,4,5-trifluorophenyl)butanoyl]-N-(3-cyanophenyl)-2,5-diazabicyclo[2.2.1]heptane-2-carboxamide and its trifluoroacetic acid salt

ESI-MS (m/z): 458.00 (M⁺+1) free amine.
Compound No. 123: (1R,4R)-5-[(3R)-3-amino-4-(2,4,5-trifluorophenyl)butanoyl]-N-(2-isopropylphenyl)-2,5-diazabicyclo[2.2.1]heptane-2-carboxamide and its trifluoroacetic acid salt

ESI-MS (m/z): 475.07 (M⁺+1) free amine.
Compound No. 124: (1R,4R)-5-[(3R)-3-amino-4-(2,4,5-trifluorophenyl)butanoyl]-N-[3,5-bis(trifluoromethyl)phenyl]-2,5-diazabicyclo[2.2.1]heptane-2-carboxamide and its trifluoroacetic acid salt

ESI-MS (m/z): 568.93 (M⁺+1) free amine.

### Example 4

### Synthesis of (2R)-4-[(1R,4R)-5-(3,5-difluorobenzyl)-2,5-diazabicyclo[2.2.1] hept-2-yl]-4-oxo-1-(2,4,5-trifluorophenyl)butan-2-amine (TFA Salt) (Compound No. 111)

### Step a: Synthesis of tert-butyl [(1R)-3-[(1S,4S)-5-(3,5-difluorobenzyl)-2,5-diazabicyclo[2.2.1]-hept-2-yl]-3-oxo-1-(2,4,5-trifluorobenzyl)propyl]carbamate

An ice-cooled solution of the compound obtained from step a of Example 3 (0.1 g, 0.27 mmol) and 3,5-difluorobenzaldehyde (0.038 g, 0.27 mmol) in dry dichloromethane (5 ml) was treated with sodiumtriacetoxyborohydride (0.17 g, 0.8 mmol) and stirred at room temperature for 20 hours. The reaction mixture was cooled to 0° C., quenched with saturated ammonium chloride solution and extracted with dichloromethane. The combined organics were washed with water, brine, dried over anhydrous sodium sulfate and concentrated under vacuo to obtain the crude product that was purified by flash column chromatography using hexane/ethyl acetate (85:15) to give the title compound (0.1 g, 69%)

ESI-MS (m/z): 540.09 (M⁺+1) (m/z).

### Step b: (2R)-4-[(1R,4R)-5-(3,5-difluorobenzyl)-2,5-diazabicyclo[2.2.1]hept-2-yl]-4-oxo-1-(2,4,5-trifluorophenyl)butan-2-amine (TFA Salt)

The compound obtained from step a (0.10 g, 0.18 mmol) was dissolved in dichloromethane (5 ml) and added trifluoroacetic acid (0.35 ml, 4.7 mmol) into it at room temperature. The reaction mixture was stirred for overnight and then concentrated. The resultant residue was stirred in diethyl ether for 10 minutes, filtered and dried to obtain the title compound (0.056 g, % yield: 70.7%)

¹H NMR (400 MHz, MeOH-d4): δ 7.32-7.34 (m, 1H), 7.21-7.24 (m, 3H), 7.08-7.20 (m, 1H), 4.6-4.80 (m, 1H), 4.2-4.5 (m, 2H), 3.75-3.90 (m, 2H), 3.45-3.63 (m, 2H), 3.04-3.3 (m, 3H), 2.45-2.9 (m, 2H), 2.25-2.42 (m, 2H), 2.05-2.2 (m, 1H);

ESI-MS (m/z): 440.05 (M⁺+1) (m/z).

### DPP IV Assay

### Materials:

H-Gly-Pro-7-amido-methylcoumarine (Gly-Pro-AMC; Cat. # G2761) and coumarine (AMC; Cat. # A9891) were purchased from Sigma. A stock solution of 1 mM Gly-Pro-AMC was prepared in 50 mM HEPES buffer, pH 7.8, containing 80 mM MgCl2, 140 mM NaCl and 1% BSA (working buffer). A solution of 1 mM AMC was prepared in 10% dimethylsulfoxide (DMSO). Aliquots were stored at −20° C.

### DPP IV Assay:

The DPP IV enzyme activity was determined using the fluorometric assay with the substrate Gly-Pro-AMC, which is cleaved by DPP IV to release the fluorescent AMC leaving group. The test compounds were dissolved in 100% dimethylsulfoxide to get a final concentration of 10 mM. The compounds were diluted serially in 10% DMSO to get 10× concentrations of 10 nM, 100 nM, 1000 nM, 10 μM, 100 μM, and 1000 μM. The source of DPP IV was human plasma, which was procured from local blood bank. DPP IV (10 μl human plasma) was mixed in 96-well FluoroNunc plates with test compounds. The final concentrations of the compounds were 1 nM, 10 nM, 100 nM, 1000 nM, 10 μM and 100 μM in working buffer, which were pre-incubated at 25° C. for 15 minutes. The assay was also carried out with 1% DMSO (final concentration), lacking the compound, as vehicle control. The reaction was started by adding 20 μl of 0.1 mM H-Gly-Pro-AMC (40 μM final concentration), followed by mixing and incubation at 25° C. for 20 minutes. The reaction was arrested by adding 50 μl of 25% acetic acid. The fluorescence was measured at an excitation filter of 380 nM and emission filter of 460 nM.

The DPP IV releases AMC from Gly-Pro-AMC, which was quantitated as relative fluorescence units (RFU). The percentage of activity was calculated as follows:

=(100/RFU of vehicle control)×RFU of test(with compound)

### IC₅₀ Determination

The IC₅₀ is defined as the concentration of the inhibitor required to inhibit 50% of the human DPP IV activity under specific assay conditions. The activity obtained at different concentrations of the compound was plotted as log (X) vs. % activity in y-axis. The IC₅₀ values were calculated using non-linear regression analysis (GradPad Prism4).

The compounds provided herein showed activity (IC₅₀) between 1 nM-10 μM following this assay, for example, from about 1900 nM to about 10.4 μM, or, for example, from about 500 nM to about 10.4 μM, or, for example, 200 nM to about 10.4 μM, or, for example, from about 75 nM to about 10.4 μM, or, for example, from about 40 nM to about 10.4 μM.

We claim:

1. Compounds having the structure of formula 1 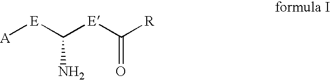
including pharmaceutically acceptable salts, pharmaceutically acceptable solvates, enantiomers, diastereomers, polymorphs, prodrugs, metabolites or N-oxides thereof,
wherein
A is an aryl or heteroaryl group;
E and E′ are independently —(CRₐR_{b})₁— (wherein 1 is an integer of 1 to 2 and Rₐ and R_{b} are independently selected from the group consisting of hydrogen, alkyl, alkenyl, alkynyl, cycloalkyl, aryl, heteroaryl or heterocyclyl; Rₐ and R_{b} can together form a ring, which can be optionally unsaturated); and
R is selected from the groups a to c: 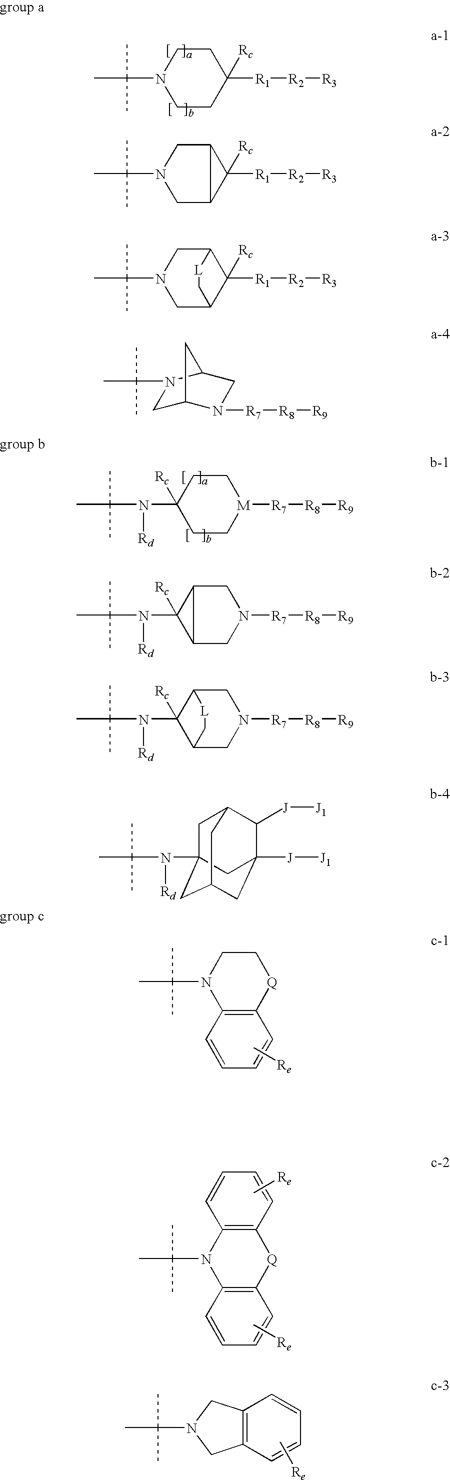
wherein
R_{c} is hydrogen or alkyl;
R_{d} is hydrogen, alkyl, aryl, heteroaryl, heterocyclyl or cycloalkyl;
Rₑ is hydrogen, alkyl, halogen, cyano, carboxy, hydroxyl, alkoxy, carbonyl or amino;
a and b are an integer of 0-2;
J is a bond, —O—, —NR_{f}—, —NR_{f}CO—, —NR_{f}CONR_{f}—, —NR_{f}SO₂—, —NR_{f}C(O)O—, or —OCONR_{f}—, wherein R_{f} refers to hydrogen, alkyl, cycloalkyl, aryl, heteroaryl or heterocyclyl;
J₁ is hydrogen, alkyl, cycloalkyl, aryl, heteroaryl or heterocyclyl (when J is —NR_{f}SO₂—, or NR_{f}C(O)O—, then J₁ is not hydrogen);
L is (CH₂)ₚ wherein p is an integer of 1-2;
M is CH or N;
Q is (CH₂)_{q}, O or S(O)_{q} wherein q is an integer of 0-2;
R₁ is —(CRₐR_{b})ₘ— wherein m is an integer of 0-1;
R₂ is —NR_{f}—, —O—, —CO—, —CS—, —CONR_{f}—, —NR_{f}CO—, —NR_{f}CONR_{f}—, —NR_{f}SO₂—, —NR_{f}COO—, or
—OCONR_{f}—; wherein R_{f} is defined as above;
R₃ is alkyl, alkenyl, alkynyl, cycloalkyl, aryl, heteroaryl or heterocyclyl;
R₇ is no atom, —CO—, —CS—, and —SO₂—;
R₈ is no atom, —O— or —NR_{f}—; and
R9 is alkyl, alkenyl, alkynyl, cycloalkyl, aryl, heteroaryl, or heterocyclyl with the following provisos:
(i) when A, E, and E′ are defined as earlier, R as a-1 and a-2 (group a), R₁ as —(CRₐR_{b})ₘ— {wherein m=0 or m=1 when a=b=1}, and R₂ as —NR_{f}—, then R₃ cannot be a heteroaryl,
(ii) when A, E, and E′ are defined as earlier, R as a-1 (group a) {wherein a is an integer of 0 and b is an integer of 1}, R₁ as —(CRₐR_{b})ₘ-{wherein m is an integer of 0}, and R₃ as defined earlier, then R₂ cannot be —CONR_{f},
(iii) when A, E, and E′ are defined as earlier, R as a-1 (group a) wherein a is an integer of 0 and b is an integer of 0-2, R₂ as —O— and —NR_{f}— and R₃ as defined earlier, then R₁ cannot be —(CRₐR_{b})ₘ— [wherein m is an integer of 1],
(iv) when A, E, and E′ are defined as earlier, R as b-1 (when M is N) and b-2 (group b) or a-4 (group a) wherein R₇ and R₈ are no atom, then R₉ cannot be a heteroaryl, and
(v) when A, E, and E′ are defined as earlier, R as a-4 (group a), b-1 (when M is N), b-2, and b-3, (group b), and R₇ as no atom, then R₈ cannot be —O— or —NR_{f}—.

2. A compound selected from the group consisting of
Compound no 1: (3R)-3-Amino-N-[1-(morpholin-4-ylcarbonyl)piperidin-4-yl]-4-(2,4,5-trifluorophenyl)butanamide and its trifluoroacetic acid salt,
Compound no 2: (3R)-3-Amino-N-{1-[(4-fluorophenyl)sulphonyl]piperidin-4-yl}-4-(2,4,5-triflu-orophenyl)butanamide and its 4-methylbenzenesulfonic acid salt,
Compound no 3: (3R)-3-Amino-N-[1-(4-fluorobenzoyl)piperidin-4-yl]-4-(2,4,5-trifluorophenyl) butanamide and its 4-methylbenzenesulfonic acid salt,
Compound no 4: N-{1-[(3R)-3-Amino-4-(2,4,5-trifluorophenyl)butanoyl]piperidin-4-yl}-4-fluorobenzamide and its trifluoroacetic acid salt,
Compound no 5: (3R)-3-Amino-N-[1-(methylsulphonyl)piperidin-4-yl]-4-(2,4,5-trifluorophenyl) butanamide and its 4-methylbenzenesulfonic acid salt,
Compound no 6: (3R)-3-Amino-N-(3-hydroxy-1-adamantyl)-4-(2,4,5-trifluorophenyl) butanamide and its 4-methylbenzenesulfonic acid salt,
Compound no 7: 4-{1[(3R)-3-Amino-4-(2,4,5-trifluorophenyl)butanoyl]amino}-N-(4-chloro phenyl)piperidine-1-carboxamide and its hydrochloride salt,
Compound no 8: (3R)-3-Amino-N-[(1R,5S)-3-(2-thienylsulphonyl)-3-azabicyclo[3.1.0]hex-6-yl]-4-(2,4,5-trifluorophenyl)butanamide and its trifluoroacetic acid salt,
Compound no 9: (3R)-3-Amino-N-[(1R,5S)-3-(4-cyanobenzoyl)-3-azabicyclo[3.1.0]hex-6-yl]-4-(2,4,5-trifluorophenyl)butanamide and its trifluoroacetic acid salt,
Compound no 10: (3R)-3-Amino-N-[(1R,5S)-3-(2,6-difluorobenzoyl)-3-azabicyclo[3.1.0]hex-6-yl]-4-(2,4,5-trifluorophenyl)butanamide and its trifluoroacetic acid salt,
Compound no. 11: N-{1-[(3R)-3-Amino-4-(2,4,5-trifluorophenyl)butanoyl]piperidin-4-yl}-4-flu-orobenzenesulfonamide and its trifluoroacetic acid salt,
Compound no. 12: N-{1-[(3R)-3-Amino-4-(2,4,5-trifluorophenyl)butanoyl]piperidin-4-yl}mor-pholine-4-carboxamide and its trifluoroacetic acid salt,
Compound no. 13: 1-{1-[(3R)-3-Amino-4-(2,4,5-trifluorophenyl)butanoyl]piperidin-4-yl}-3-(4-chlorophenyl)urea and its trifluoroacetic acid salt,
Compound no. 14: N-{1-[(3R)-3-Amino-4-(2,4,5-trifluorophenyl)butanoyl]piperidin-4-yl}-3-fluoro-4-methoxybenzamide and its trifluoroacetic acid salt,
Compound no. 15: N-{1-[(3R)-3-Amino-4-(2,4,5-trifluorophenyl)butanoyl]piperidin-4-yl}-2-propanesulfonamide and its trifluoroacetic acid salt,
Compound no 16: (3R)-3-Amino-N-[(1R,5S)-3-(4-trifluorobenzenesulphonyl)-3-azabicyclo [3.1.0]hex-6-yl]-4-(2,4,5-trifluorophenyl)butanamide and its trifluoroacetic acid salt,
Compound no 17: (3R)-3-Amino-N-[(1R,5S)-3-(thiophene-2-carbonyl)-3-azabicyclo[3.1.0]hex-6-yl]-4-(2,4,5-trifluorophenyl)butanamide and its trifluoroacetic acid salt,
Compound no 18: (3R)-3-Amino-N-[(1R,5S)-3-(ethanesulphonyl)-3-azabicyclo[3.1.0]hex-6-yl]-4-(2,4,5-trifluorophenyl)butanamide and its trifluoroacetic acid salt,
Compound no 19: (3R)-3-Amino-N-[(1R,5S)-3-(4-methylbenznesulphonyl)-3-azabicyclo[3.1.0] hex-6-yl]-4-(2,4,5-trifluorophenyl)butanamide and its trifluoroacetic acid salt,
Compound no 20: 4-[({(3R)-1-[(3R)-3-Amino-4-(2,4,5-trifluorophenyl)butanoyl]pyrrolidin-3-yl}oxy)methyl]benzonitrile and its trifluoroacetic acid salt,
Compound no 21: (2R)-4-{(3S)-3-[(4-Fluorobenzyl)oxy]pyrrolidin-1-yl}-4-oxo-1-(2,4,5-trifluorophenyl)butan-2-amine and its trifluoroacetic acid salt,
Compound no 22: 4-[({(3S)-1-[(3R)-3-Amino-4-(2,4,5-trifluorophenyl)butanoyl]pyrrolidin-3-yl}oxy)methyl]benzonitrile and its trifluoroacetic acid salt,
Compound No. 23: N-{1-[(3R)-3-amino-4-(2,4,5-trifluorophenyl)butanoyl]piperidin-4-yl}-2,2,2-trifluoroethanesulfonamide and its trifluoroacetic acid salt,
Compound No. 24: N-{1-[(3R)-3-amino-4-(2,4,5-trifluorophenyl)butanoyl]piperidin-4-yl}-4-cyanobenzenesulfonamide and its trifluoroacetic acid salt,
Compound No. 25: N-{1-[3-amino-4-(2,4,5-trifluorophenyl)butanoyl]piperidin-4-yl}-2,4-difluorobenzenesulfonamide and its trifluoroacetic acid salt,
Compound No. 26: Methyl 5-[({1-[(3R)-3-amino-4-(2,4,5-trifluorophenyl)butanoyl]piperidin-4-yl} amino)sulfonyl]-4-methylthiophene-2-carboxylate,
Compound No. 27: N-{1-[(3R)-3-amino-4-(2,4,5-trifluorophenyl)butanoyl]piperidin-4-yl}-1,3,5-trimethyl-1H-pyrazole-4-sulfonamide,
Compound No. 28: methyl 4-[({1-[(3R)-3-amino-4-(2,4,5-trifluorophenyl)butanoyl]piperidin-4-yl} amino)sulfonyl]-2,5-dimethyl-3-furoate,
Compound No. 29: N-{1-[(3R)-3-amino-4-(2,4,5-trifluorophenyl)butanoyl]piperidin-4-yl}-4-fluorobenzenesulfonamide,
Compound No. 30: 4-acetyl-N-{1-[(3R)-3-amino-4-(2,4,5-trifluorophenyl)butanoyl] piperidin-4-yl}benzenesulfonamide and its trifluoroacetic acid salt,
Compound No. 31: N-{1-[(3R)-3-amino-4-(2,4,5-trifluorophenyl)butanoyl]piperidin-4-yl}-2,4-difluorobenzenesulfonamide and its trifluoroacetic acid salt,
Compound No. 32: N-{1-[(3R)-3-amino-4-(2,4,5-trifluorophenyl)butanoyl]piperidin-4-yl} methane sulfonamide and its trifluoroacetic acid salt,
Compound No. 33: methyl 5-[({1-[(3R)-3-amino-4-(2,4,5-trifluorophenyl)butanoyl] piperidin-4-yl} amino) sulfonyl]-4-methylthiophene-2-carboxylate and its trifluoroacetic acid salt,
Compound No. 34: N-{1-[(3R)-3-amino-4-(2,4,5-trifluorophenyl)butanoyl]piperidin-4-yl} propane-2-sulfonamide and its trifluoroacetic acid salt,
Compound No. 35: N-{(3S)-1-[(3R)-3-amino-4-(2,4,5-trifluorophenyl)butanoyl]piperidin-3-yl}ethane sulfonamide and its trifluoroacetic acid salt,
Compound No. 36: 4-(1,3-dihydro-2H-isoindol-2-yl)-4-oxo-1-(2,4,5-trifluorophenyl)butan-2-amine and its trifluoroacetic acid salt,
Compound No. 37: N-{1-[3-amino-4-(2,4,5-trifluorophenyl)butanoyl]piperidin-4-yl}propanamide and its trifluoroacetic acid salt,
Compound No. 38: N-{1-[(3R)-3-amino-4-(2,4,5-trifluorophenyl)butanoyl]piperidin-4-yl}acetamide and its trifluoroacetic acid salt,
Compound No. 39: N-{1-[(3R)-3-amino-4-(2,4,5-trifluorophenyl)butanoyl]piperidin-4-yl}-4-fluorobenzamide,
Compound No. 40: 2-({1-[(3R)-3-amino-4-(2,4,5-trifluorophenyl)butanoyl]piperidin-4-yl} amino)-4,6-difluorobenzonitrile and its trifluoroacetic acid salt,
Compound No. 41: 2-({1-[(3R)-3-amino-4-(2,4,5-trifluorophenyl)butanoyl]piperidin-4-yl} amino)benzonitrile and its trifluoroacetic acid salt,
Compound No. 42: 4-({1-[(3R)-3-amino-4-(2,4,5-trifluorophenyl)butanoyl]piperidin-4-yl} amino)-2,6-difluorobenzonitrile and its 4-methylbenzenesulfonic acid salt,
Compound No. 43: 4-({1-[(3R)-3-amino-4-(2,4,5-trifluorophenyl)butanoyl]piperidin-4-yl} amino)-2,6-difluorobenzonitrile and its trifluoroacetic acid salt,
Compound No. 44: 2-({1-[(3R)-3-amino-4-(2,4,5-trifluorophenyl)butanoyl]piperidin-4-yl} amino)benzonitrile and its trifluoroacetic acid salt,
Compound No. 45: 4-({1-[(3R)-3-amino-4-(2,4,5-trifluorophenyl)butanoyl]piperidin-4-yl} amino)benzonitrile and its trifluoroacetic acid salt,
Compound No. 46: 4-({1-[(3R)-3-amino-4-(2,4,5-trifluorophenyl)butanoyl]piperidin-4-yl} amino)benzonitrile and its trifluoroacetic acid salt,
Compound No. 47: 4-({1-[(3R)-3-amino-4-(2,4,5-trifluorophenyl)butanoyl]piperidin-4-yl} amino)-2-(trifluoromethyl)benzonitrile and its trifluoroacetic acid salt,
Compound No. 48: Ethyl ({(3S)-1-[(3R)-3-amino-4-(2,4,5-trifluorophenyl)butanoyl]pyrrolidin-3-yl}oxy)acetate and its trifluoroacetic acid salt,
Compound No. 49: (2R)-4-oxo-4-[5-(2-thienylacetyl)-2,5-diazabicyclo[2.2.1]hept-2-yl]-1-(2,4,5-trifluorophenyl)butan-2-amine and its trifluoroacetic acid salt,
Compound No. 50: (2R)-4-oxo-4-[5-(trifluoroacetyl)-2,5-diazabicyclo[2.2.1]hept-2-yl]-1-(2,4,5-trifluorophenyl)butan-2-amine and its hydrochloride salt,
Compound No. 51: (2R)-4-oxo-4-(5-propionyl-2,5-diazabicyclo[2.2.1]hept-2-yl)-1-(2,4,5-trifluoro phenyl) butan-2-amine and its hydrochloride salt,
Compound No. 52: 5-[(3R)-3-amino-4-(2,4,5-trifluorophenyl)butanoyl]-N-(4-cyanophenyl)-2,5-diazabicyclo[2.2.1]heptane-2-carboxamide and its 4-methylbenzenesulfonic acid salt,
Compound No. 53: (2R)-4-(5-acetyl-2,5-diazabicyclo[2.2.1]hept-2-yl)-4-oxo-1-(2,4,5-trifluoro-phenyl)butan-2-amine and its trifluoroacetic acid salt,
Compound No. 54: (1S,4S)-5-[(3R)-3-amino-4-(2,4,5-trifluorophenyl)butanoyl]-N-(4-fluoro-phenyl)-2,5-diazabicyclo[2.2.1]heptane-2-carboxamide and its trifluoroacetic acid salt,
Compound No. 55: (1S,4S)-5-[(3R)-3-amino-4-(2,4,5-trifluorophenyl)butanoyl]-N-(4-methoxy-phenyl)-2,5-diazabicyclo[2.2.1]heptane-2-carboxamide and its trifluoroacetic acid salt,
Compound No. 56: (1S,4S)-5-[(3R)-3-amino-4-(2,4,5-trifluorophenyl)butanoyl]-N-[4-(trifluoro-methyl)phenyl]-2,5-diazabicyclo[2.2.1]heptane-2-carboxamide and its trifluoroacetic acid salt,
Compound No. 57: (1S,4S)-5-[(3R)-3-amino-4-(2,4,5-trifluorophenyl)butanoyl]-N-benzyl-2,5-diazabicyclo[2.2.1]heptane-2-carboxamide and its trifluoroacetic acid salt,
Compound No. 58: (1S,4S)-5-[(3R)-3-amino-4-(2,4,5-trifluorophenyl)butanoyl]-N-(4-methyl-phenyl)-2,5-diazabicyclo[2.2.1]heptane-2-carboxamide and its trifluoroacetic acid salt,
Compound No. 59: (1S,4S)-5-[(3R)-3-amino-4-(2,4,5-trifluorophenyl)butanoyl]-N-tert-butyl-2,5-diaza-bicyclo[2.2.1]heptane-2-carboxamide and its trifluoroacetic acid salt,
Compound No. 60: (2R)-4-{5-[(3-fluorophenyl)sulfonyl]-2,5-diazabicyclo[2.2.1]hept-2-yl}-4-oxo-1-(2,4,5-trifluorophenyl)butan-2-amine and its trifluoroacetic acid salt,
Compound No. 61: (1S,4S)-5-[(3R)-3-amino-4-(2,4,5-trifluorophenyl)butanoyl]-N-(3-fluoro-phenyl)-2,5-diazabicyclo[2.2.1]heptane-2-carboxamide and its trifluoroacetic acid salt,
Compound No. 62: (1S,4S)-5-[(3R)-3-amino-4-(2,4,5-trifluorophenyl)butanoyl]-N-[2-(trifluoromethyl)phenyl]-2,5-diazabicyclo[2.2.1]heptane-2-carboxamide and its trifluoroacetic acid salt,
Compound No. 63: (1S,4S)-5-[(3R)-3-amino-4-(2,4,5-trifluorophenyl)butanoyl]-N-(2-methyl-phenyl)-2,5-diazabicyclo[2.2.1]heptane-2-carboxamide and its trifluoroacetic acid salt,
Compound No. 64: (1S,4S)-5-[(3R)-3-amino-4-(2,4,5-trifluorophenyl)butanoyl]-N-(4-nitro-phenyl)-2,5-diazabicyclo[2.2.1]heptane-2-carboxamide and its trifluoroacetic acid salt,
Compound No. 65: 4-{(1S,4S)-5-[(3R)-3-amino-4-(2,4,5-trifluorophenyl)butanoyl]-2,5-diaza-bicyclo[2.2.1]hept-2-yl}-2-chlorobenzonitrile and its trifluoroacetic acid salt,
Compound No. 66: 2-{(1S,4S)-5-[(3R)-3-amino-4-(2,4,5-trifluorophenyl)butanoyl]-2,5-diaza-bicyclo[2.2.1]hept-2-yl}-6-fluorobenzonitrile and its trifluoroacetic acid salt,
Compound No. 67: 4-{(1S,4S)-5-[(3R)-3-amino-4-(2,4,5-trifluorophenyl)butanoyl]-2,5-diaza-bicyclo[2.2.1]hept-2-yl}-3-fluorobenzonitrile and its trifluoroacetic acid salt,
Compound No. 68: (1S,4S)-5-[(3R)-3-amino-4-(2,4,5-trifluorophenyl)butanoyl]-N-cyclohexyl-2,5-diazabicyclo[2.2.1]heptane-2-carboxamide and its trifluoroacetic acid salt,
Compound No. 69: (1S,4S)-5-[(3R)-3-amino-4-(2,4,5-trifluorophenyl)butanoyl]-N-(3-methoxy-phenyl)-2,5-diazabicyclo[2.2.1]heptane-2-carboxamide and its trifluoroacetic acid salt,
Compound No. 70: (1S,4S)-5-[(3R)-3-amino-4-(2,4,5-trifluorophenyl) butanoyl]-N-(2-fluoro-phenyl)-2,5-diazabicyclo[2.2.1]heptane-2-carboxamide and its trifluoroacetic acid salt,
Compound No. 71: (2R)-4-[5-(ethylsulfonyl)-2,5-diazabicyclo[2.2.1]hept-2-yl]-4-oxo-1-(2,4,5-trifluorophenyl)butan-2-amine and its trifluoroacetic acid salt,
Compound No. 72: (1S,4S)-5-[(3R)-3-amino-4-(2,4,5-trifluorophenyl)butanoyl]-N-(2,4-dimethoxyphenyl)-2,5-diazabicyclo[2.2.1]heptane-2-carboxamide and its trifluoroacetic acid salt,
Compound No. 73: (1S,4S)-5-[(3R)-3-amino-4-(2,4,5-trifluorophenyl)butanoyl]-N-isopropyl-2,5-diazabicyclo[2.2.1]heptane-2-carboxamide and its trifluoroacetic acid salt,
Compound No. 74: 4-{5-[(3R)-3-amino-4-(2,4,5-trifluorophenyl)butanoyl]-2,5-diazabicyclo-[2.2.1]hept-2-yl}-2-(trifluoromethyl)benzonitrile and its trifluoroacetic acid salt,
Compound No. 75: (1S,4S)-5-[(3R)-3-amino-4-(2,4,5-trifluorophenyl)butanoyl]-N-[4-(benzyl-oxy)phenyl]-2,5-diazabicyclo[2.2.1]heptane-2-carboxamide and its trifluoroacetic acid salt,
Compound No. 76: (2R)-4-[5-(4-fluorobenzoyl)-2,5-diazabicyclo[2.2.1]hept-2-yl]-4-oxo-1-(2,4,5-trifluorophenyl)butan-2-amine and its trifluoroacetic acid salt,
Compound No. 77: (1S,4S)-5-[(3R)-3-amino-4-(2,4,5-trifluorophenyl)butanoyl]-N-(3,4,5-tri-methoxyphenyl)-2,5-diazabicyclo[2.2.1]heptane-2-carboxamide and its trifluoroacetic acid salt,
Compound No. 78: 5-[3-amino-4-(2,4,5-trifluorophenyl)butanoyl]-N-(4-fluorophenyl)-2,5-diaza-bicyclo[2.2.1]heptane-2-carboxamide and its trifluoroacetic acid salt,
Compound No. 79: (1S,4S)-5-[3-amino-4-(2,4,5-trifluorophenyl)butanoyl]-N-(2,6-diisopropyl phenyl)-2,5-diazabicyclo[2.2.1]heptane-2-carboxamide and its trifluoroacetic acid salt,
Compound No. 80: methyl 2-[({(1S,4S)-5-[(3R)-3-amino-4-(2,4,5-trifluorophenyl)butanoyl]-2,5-diaza-bicyclo[2.2.1]hept-2-yl}carbonyl)amino]benzoate and its trifluoroacetic acid salt,
Compound No. 81: (2R)-4-oxo-4-[5-(2-thienylsulfonyl)-2,5-diaza-bicyclo[2.2.1]hept-2-yl]-1-(2,4,5-trifluorophenyl)butan-2-amine and its trifluoroacetic acid salt,
Compound No. 82: (1S,4S)-5-[(3R)-3-amino-4-(2,4,5-trifluorophenyl)butanoyl]-N-(5-chloro-2-methoxy phenyl)-2,5-diazabicyclo [2.2.1]heptane-2-carboxamide and its trifluoroacetic acid salt,
Compound No. 83: 4-({5-[(3R)-3-amino-4-(2,4,5-trifluoro phenyl)butanoyl]-2,5-diazabicyclo [2.2.1]hept-2-yl}sulfonyl)benzonitrile and its trifluoroacetic acid salt,
Compound No. 84: (1S,4S)-5-[(3R)-3-amino-4-(2,4,5-trifluorophenyl)butanoyl]-N-(2,3-dichlorophenyl)-2,5-diazabicyclo [2.2.1]heptane-2-carboxamide and its trifluoroacetic acid salt,
Compound No. 85: (2R)-4-{(1S,4S)-5-[(3,5-difluorophenyl) sulfonyl]-2,5-diazabicyclo [2.2.1]hept-2-yl}-4-oxo-1-(2,4,5-trifluorophenyl) butan-2-amine and its trifluoroacetic acid salt,
Compound No. 86: (1S,4S)—N-(4-acetylphenyl)-5-[(3R)-3-amino-4-(2,4,5-trifluorophenyl) butanoyl]-2,5-diazabicyclo[2.2.1]heptane-2-carboxamide and its trifluoroacetic acid salt,
Compound No. 87: methyl 5-[(3R)-3-amino-4-(2,4,5-trifluorophenyl)butanoyl]-2,5-diazabicyclo [2.2.1]heptane-2-carboxylate and its trifluoroacetic acid salt,
Compound No. 88: (1S,4S)-5-[(3R)-3-amino-4-(2,4,5-trifluorophenyl)butanoyl]-N-(2,5-dimethoxyphenyl)-2,5-diazabicyclo[2.2.1]heptane-2-carboxamide and its trifluoroacetic acid salt,
Compound No. 89: ethyl 5-[(3R)-3-amino-4-(2,4,5-trifluorophenyl)butanoyl]-2,5-diazabicyclo [2.2.1]heptane-2-carboxylate and its trifluoroacetic acid salt,
Compound No. 90: (2R)-4-oxo-4-[5-(propylsulfonyl)-2,5-diazabicyclo[2.2.1]hept-2-yl]-1-(2,4,5-trifluoro-phenyl)butan-2-amine and its trifluoroacetic acid salt,
Compound No. 91: (2R)-4-[5-(isopropylsulfonyl)-2,5-diazabicyclo[2.2.1]hept-2-yl]-4-oxo-1-(2,4,5-trifluoro phenyl)butan-2-amine and its trifluoroacetic acid salt,
Compound No. 92: (2R)-4-[5-(butylsulfonyl)-2,5-diazabicyclo[2.2.1]hept-2-yl]-4-oxo-1-(2,4,5-trifluoro-phenyl)butan-2-amine and its trifluoroacetic acid salt,
Compound No. 93: (2R)-4-oxo-4-[5-(phenylsulfonyl)-2,5-diazabicyclo[2.2.1]hept-2-yl]-1-(2,4,5-trifluorophenyl)butan-2-amine and its trifluoroacetic acid salt,
Compound No. 94: (2R)-4-[5-(morpholin-4-ylcarbonyl)-2,5-diazabicyclo[2.2.1]hept-2-yl]-4-oxo-1-(2,4,5-trifluorophenyl)butan-2-amine and its trifluoroacetic acid salt,
Compound No. 95: (2R)-4-oxo-4-(5-{[3-(trifluoromethoxy)phenyl]sulfonyl}-2,5-diazabicyclo [2.2.1]hept-2-yl)-1-(2,4,5-trifluorophenyl)butan-2-amine and its trifluoroacetic acid salt,
Compound No. 96: (2R)-4-[5-(methylsulfonyl)-2,5-diazabicyclo[2.2.1]hept-2-yl]-4-oxo-1-(2,4,5-trifluorophenyl)butan-2-amine and its trifluoroacetic acid salt,
Compound No. 97: (1S,4S)-5-[(3R)-3-amino-4-(2,4,5-trifluorophenyl)butanoyl]-N-(2,6-difluoro-phenyl)-2,5-diazabicyclo [2.2.1]heptane-2-carboxamide and its trifluoroacetic acid salt,
Compound No. 98: (1S,4S)-5-[(3R)-3-amino-4-(2,4,5-trifluorophenyl)butanoyl]-N-(2,4,6-trifluoro-phenyl)-2,5-diazabicyclo[2.2.1]heptane-2-carboxamide and its trifluoroacetic acid salt,
Compound No. 99: (1S,4S)—N-(3-acetylphenyl)-5-[(3R)-3-amino-4-(2,4,5-trifluorophenyl) butanoyl]-2,5-diazabicyclo[2.2.1]heptane-2-carboxamide and its trifluoroacetic acid salt,
Compound No. 100: (1S,4S)-5-[(3R)-3-amino-4-(2,4,5-trifluorophenyl)butanoyl]-N-(2,5-dichloro-phenyl)-2,5-diazabicyclo[2.2.1]heptane-2-carboxamide and its trifluoroacetic acid salt,
Compound No. 101: (1S,4S)-5-[(3R)-3-amino-4-(2,4,5-trifluorophenyl)butanoyl]-N-(4-isopropyl-phenyl)-2,5-diazabicyclo[2.2.1]heptane-2-carboxamide and its trifluoroacetic acid salt,
Compound No. 102: (1S,4S)-5-[(3R)-3-amino-4-(2,4,5-trifluorophenyl)butanoyl]-N-(4-butyl-phenyl)-2,5-diazabicyclo[2.2.1]heptane-2-carboxamide and its trifluoroacetic acid salt,
Compound No. 103: (1S,4S)-5-[(3R)-3-amino-4-(2,4,5-trifluorophenyl)butanoyl]-N-(4-ethoxy-phenyl)-2,5-diazabicyclo[2.2.1]heptane-2-carboxamide and its trifluoroacetic acid salt,
Compound No. 104: (1S,4S)-5-[(3R)-3-amino-4-(2,4,5-trifluorophenyl)butanoyl]-N-(3-ethyl-phenyl)-2,5-diazabicyclo[2.2.1]heptane-2-carboxamide and its trifluoroacetic acid salt,
Compound No. 105: (1S,4S)-5-[(3R)-3-amino-4-(2,4,5-trifluorophenyl)butanoyl]-N-(2-isopropyl-6-methylphenyl)-2,5-diazabicyclo[2.2.1]heptane-2-carboxamide and its trifluoroacetic acid salt,
Compound No. 106: (1S,4S)-5-[(3R)-3-amino-4-(2,4,5-trifluorophenyl)butanoyl]-N-mesityl-2,5-diazabicyclo[2.2.1]heptane-2-carboxamide and its trifluoroacetic acid salt,
Compound No. 107: (1S,4S)-5-[(3R)-3-amino-4-(2,4,5-trifluorophenyl)butanoyl]-N-(4-methoxy-2-methylphenyl)-2,5-diazabicyclo[2.2.1]heptane-2-carboxamide and its trifluoroacetic acid salt,
Compound No. 108: (1S,4S)-5-[(3R)-3-amino-4-(2,4,5-trifluorophenyl)butanoyl]-N-(4-phenoxy-phenyl)-2,5-diazabicyclo[2.2.1]heptane-2-carboxamide and its trifluoroacetic acid salt,
Compound No. 109: (2R)-4-[(1R,4R)-5-(cyclohexylcarbonyl)-2,5-diazabicyclo[2.2.1]hept-2-yl]-4-oxo-1-(2,4,5-trifluorophenyl) butan-2-amine and its trifluoroacetic acid salt,
Compound No. 110: (2R)-4-[(1R,4R)-5-(cyclopropyl carbonyl)-2,5-diazabicyclo [2.2.1]hept-2-yl]-4-oxo-1-(2,4,5-trifluorophenyl) butan-2-amine and its trifluoroacetic acid salt,
Compound No. 111: (2R)-4-[(1R,4R)-5-(3,5-difluorobenzyl)-2,5-diazabicyclo[2.2.1]hept-2-yl]-4-oxo-1-(2,4,5-trifluorophenyl)butan-2-amine and its trifluoroacetic acid salt,
Compound No. 112: (1R,4R)-5-[(3R)-3-amino-4-(2,4,5-trifluorophenyl)butanoyl]-N-(2-ethoxyphenyl)-2,5-diazabicyclo[2.2.1]heptane-2-carboxamide and its trifluoroacetic acid salt,
Compound No. 113: (1R,4R)-5-[(3R)-3-amino-4-(2,4,5-trifluorophenyl)butanoyl]-N-(2-ethylphenyl)-2,5-diazabicyclo[2.2.1]heptane-2-carboxamide and its trifluoroacetic acid salt,
Compound No. 114: methyl 3-[({(1R,4R)-5-[(3R)-3-amino-4-(2,4,5-trifluorophenyl)butanoyl]-2,5-diazabicyclo[2.2.1]hept-2-yl}carbonyl)amino]benzoate and its trifluoroacetic acid salt,
Compound No. 115: (1R,4R)-5-[(3R)-3-amino-4-(2,4,5-trifluorophenyl)butanoyl]-N-(4-ethylphenyl)-2,5-diazabicyclo[2.2.1]heptane-2-carboxamide and its trifluoroacetic acid salt,
Compound No. 116: (1R,4R)-5-[(3R)-3-amino-4-(2,4,5-trifluorophenyl)butanoyl]-N-(3-chloro-4-methylphenyl)-2,5-diazabicyclo[2.2.1]heptane-2-carboxamide and its trifluoroacetic acid salt,
Compound No. 117: (1R,4R)-5-[(3R)-3-amino-4-(2,4,5-trifluorophenyl)butanoyl]-N-[3-(methylthio)phenyl]-2,5-diazabicyclo[2.2.1]heptane-2-carboxamide and its trifluoroacetic acid salt,
Compound No. 118: (1R,4R)-5-[(3R)-3-amino-4-(2,4,5-trifluorophenyl)butanoyl]-N-(2,4-difluorophenyl)-2,5-diazabicyclo[2.2.1]heptane-2-carboxamide and its trifluoroacetic acid salt,
Compound No. 119: (1R,4R)-5-[(3R)-3-amino-4-(2,4,5-trifluorophenyl)butanoyl]-N-(2,4-dimethylphenyl)-2,5-diazabicyclo[2.2.1]heptane-2-carboxamide and its trifluoroacetic acid salt,
Compound No. 120: (1R,4R)-5-[(3R)-3-amino-4-(2,4,5-trifluorophenyl)butanoyl]-N-(3,4-dichlorophenyl)-2,5-diazabicyclo[2.2.1]heptane-2-carboxamide and its trifluoroacetic acid salt,
Compound No. 121: (1R,4R)-5-[(3R)-3-amino-4-(2,4,5-trifluorophenyl)butanoyl]-N-[4-chloro-3-(trifluoromethyl)phenyl]-2,5-diazabicyclo [2.2.1]heptane-2-carboxamide and its trifluoroacetic acid salt,
Compound No. 122: (1R,4R)-5-[(3R)-3-amino-4-(2,4,5-trifluorophenyl)butanoyl]-N-(3-cyanophenyl)-2,5-diazabicyclo[2.2.1]heptane-2-carboxamide and its trifluoroacetic acid salt,
Compound No. 123: (1R,4R)-5-[(3R)-3-amino-4-(2,4,5-trifluorophenyl)butanoyl]-N-(2-isopropylphenyl)-2,5-diazabicyclo[2.2.1]heptane-2-carboxamide and its trifluoroacetic acid salt,
Compound No. 124: (1R,4R)-5-[(3R)-3-amino-4-(2,4,5-trifluorophenyl)butanoyl]-N-[3,5-bis-(trifluoromethyl)phenyl]-2,5-diazabicyclo[2.2.1]heptane-2-carboxamide and its trifluoroacetic acid salt.

3. A pharmaceutical composition comprising a therapeutically effective amount of a compound as defined in claim 1 together with a pharmaceutically acceptable carrier, excipients or diluents.

4. A pharmaceutical composition of claim 3 further comprising one or more additional active ingredients selected from the group consisting of:
i) antihyperglycaemic agents (a) other DPP IV inhibitors, e.g., saxagliptin, (b) insulin sensitizers, (I) PPAR agonists, for example, PPARγ agonists (e.g., rosiglitazone and pioglitazone), (II) PPARα/γ dual agonists (e.g., tesaglitazar and muraglitazar), and (III) PPAR pan-agonists (e.g., GSK 667954) (c) biguanides, e.g., metforminutes, (d) insulin secretagogues, for example, sulphonyl ureas (e.g., glimeperide) and non-sulphonyl ureas (e.g., repaglinide), (e) α-glucosidase inhibitors, e.g., acarbose, (f) protein tyrosine phosphatase-1B inhibitors, (g) glucokinase activators, e.g. PSN105 (h) inhibitors of 11β-hydroxysteroid dehydrogenase type 1, (i) glucagon receptor antagonists, (j) GLP-1 and GLP-1 receptor agonists, e.g. liraglutide (k) insulin or insulin mimetics, (1) GIP and GIP receptor agonists (m) PACAP and PACAP receptor agonists, (n) fructose 1,6 bisphosphatase inhibitors; (o) glucose 6 phosphatase inhibitors; (p) Sodium glucose transporter 2 (SGLT2) inhibitor, e.g., Kissei-869682; (q) AMP-Activated protein kinase activators;
ii) lipid modulating agents, (i) HMG-CoA reductase inhibitors, e.g., atorvastatin, simvastatin, fluvastatin etc. (ii) sequestrants (cholestyramine, colestipol, and dialkylaminoalkyl derivatives of a cross-linked dextran) (iii) nicotinyl alcohol, nicotinic acid or a salt thereof, (iv) inhibitors of cholesterol absorption, e.g., β-sitosterol and ezetimibe, (v) acyl CoA:cholesterol acyltransferase inhibitors, e.g., avasimibe (vi) ileal bile acid transporter inhibitors, and (vi) CETP inhibitors, e.g., torcetrapib;
iii) antiobesity compounds, (i) CB1 receptor inverse agonists and antagonists, e.g., rimonabant (ii) β3 adrenergic receptor agonists, (iii) melanocortin-receptor agonists, in particular, melanocortin-4 receptor agonists, (iv) ghrelin antagonists, (v) neuropeptide Y1 or Y5 antagonists, (vi) melanin-concentrating hormone (MCH) receptor antagonists and (vii) fenfluramine, dexfenfluramine, phentermine, sibutramine, orlistat, etc;
iv) antihypertensive agents, (i) ACE inhibitors, e.g., enalapril, lisinopril, and quinapril, (ii) angiotensin II receptor antagonists and agonists, e.g., losartan, candesartan, irbesartan, valsartan, and eprosartan, (iii) β-blockers, and (iv) calcium channel blockers; and
anti-TNF agent or c-AMP raising agent like PDE inhibitors.

5. A method for treating conditions mediated by DPP IV, selected from diabetes, type 2 diabetes, prediabetes, diabetic dyslipidemia, metabolic acidosis, ketosis, satiety disorders, and obesity; inflammatory disease, multiple sclerosis, rheumatoid arthritis; viral, cancer and gastrointestinal disorders and treatment of infertility arising due to polycystic ovary syndrome, which comprises administering to the mammal an effective amount of a compound of claim 1.

6. A method for treating conditions selected from diabetes, type 2 diabetes, prediabetes, diabetic dyslipidemia, metabolic acidosis, ketosis, satiety disorders, and obesity; inflammatory disease, multiple sclerosis, rheumatoid arthritis; viral, cancer and gastrointestinal disorders and treatment of infertility arising due to polycystic ovary syndrome, which comprises administering to the mammal an effective amount of a pharmaceutical composition according to the claim 3.

7. A method for treating conditions selected from diabetes, type 2 diabetes, prediabetes, diabetic dyslipidemia, metabolic acidosis, ketosis, satiety disorders, and obesity; inflammatory disease, multiple sclerosis, rheumatoid arthritis; viral, cancer and gastrointestinal disorders and treatment of infertility arising due to polycystic ovary syndrome, which comprises administering to the mammal an effective amount of pharmaceutical composition according to claim 4.

8. A process for preparing a compound of formula XIII, comprising
reaction of compound of formula VI 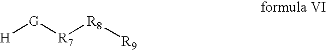
with a compound of formula XI 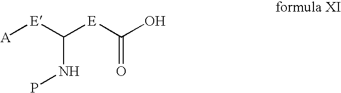
to give a compound of formula XII; and 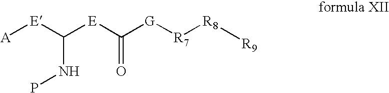
Deprotection of the compound of formula XII to give a compound of formula XIII

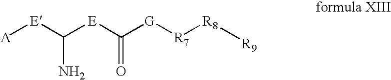

9. A process for preparing a compound of formula XIV, comprising
reaction of compound of formula VI 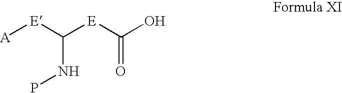
with a compound of formula XI 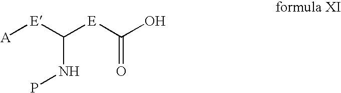
to give a compound of formula XIV; and 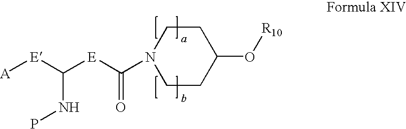
Deprotection of the compound of formula XIV to give a compound of formula XV.

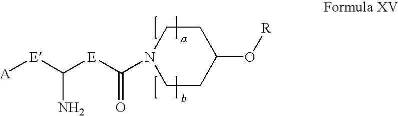

10. A process for preparing a compound of formula XX, comprising:
reacting the compound of formula Xc 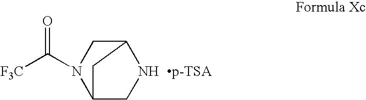
with a compound of formula XI 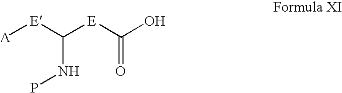
to give a compound of formula XVI; 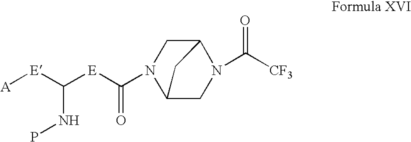
deprotecting the compound of formula XVI to give a compound of formula XVII; 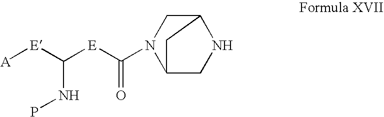
Reacting the compound of formula XVII with either a compound of formula III, formula XVIII or formula V 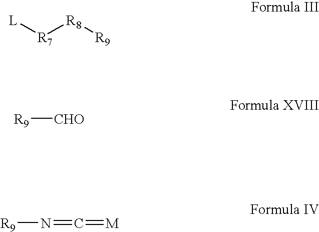
to give a compound of formula XIX; and 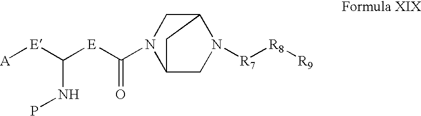
deprotecting the compound of formula XIX to yield the compound of formula XX. [chemical structure]
